(19) United States
(12) Patent Application Publication
(10) Pub. No.: US 2026/0159550 A1
(43) Pub. Date: Jun. 11, 2026

(54) Controllable Intein Splicing and N-Terminal Cleavage at Mesophilic Temperatures

(71) Applicants: Murray State University, Murray, KY (US); The Ohio State University, Columbus, OH (US)

(72) Inventors: Christopher W. Lennon, Murray, KY (US); David W. Wood, Dublin, OH (US)

(21) Appl. No.: 19/413,466

(22) Filed: Dec. 9, 2025

Related U.S. Application Data
(60) Provisional application No. 63/729,996, filed on Dec. 10, 2024.

(51) Int. Cl. C07K 14/00; C07K 1/16

(52) U.S. Cl. CPC C07K 14/00; C07K 1/16; C07K 2319/20

(57) ABSTRACT

The present disclosure related to variant polypeptides having intein activity, for example, controllable splicing and/or N-terminal cleavage, fusion proteins, comprising the variants, systems comprising the polypeptides or fusion proteins, and related kits and processes. The variants, fusion proteins, and systems can be used for a variety of applications in which intein splicing and/or N-terminal cleavage is desirable, for instance, intein-based protein expression and purification.

### RELATED APPLICATION INFORMATION

This application claims priority to U.S. Application No. 63/729,996, filed on Dec. 10, 2024, the contents of which are herein incorporated by reference.

### GOVERNMENT FUNDING INFORMATION

This invention was made with government support under P20GM103436 and 1R15GM143662-01 awarded by KY IDeA Networks of Biomedical Research Excellence and the National Institutes of Health. The government has certain rights in the invention.

### FIELD OF THE INVENTION

The present disclosure relates to polypeptides having intein activity (e.g., are capable of temperature controllable splicing and N-terminal cleavage in the absence of an external nucleophile). The present disclosure also relates to fusion proteins comprising the polypeptides, intein-based expression and purification systems comprising the polypeptides and fusion proteins, and related kits and processes.

### SEQUENCE LISTING STATEMENT

The contents of the electronic sequence listing titled KSTC_43952_202_SequenceListing.xml (Size: 5,505 bytes; and Date of Creation: Dec. 9, 2025) are herein incorporated by reference in their entirety.

### BACKGROUND OF THE INVENTION

Inteins, or intervening proteins, are mobile genetic elements removed from host proteins through protein splicing. In this process, the intein removes itself by rearranging two peptide bonds and rejoining the flanking sequences, termed N- and C-exteins, with a new peptide bond. In the canonical (class 1) mechanism of protein splicing, the reaction proceeds in four steps (FIG. 1A; reviewed in Mills et al 2014, Wood et al. 2023). First, a nucleophilic attack by the first residue of the intein (cysteine or serine) on the preceding peptide bond forms a linear (thio) ester. Second, a cysteine, serine, or threonine at the first position of the C-extein performs a second nucleophilic attack on the (thio) ester from step 1, forming a branched intermediate. Third, the last residue of the intein, an asparagine, cyclizes to release the intein. Fourth, the (thio) ester linking the N- and C-exteins rearranges to form a peptide bond, generating the uninterrupted host protein (ligated exteins).

The ability of inteins to rearrange peptide bonds in a specific and controlled manner has been exploited by engineers to develop numerous technologies, including platforms for protein purification, segmental isotope labeling, formation of cyclic peptides, incorporation of non-natural modules into proteins, fabrication of protein arrays, sensor development, imaging, and regulation of protein function in vivo (reviewed in Wood and Camarero 2014; Sarmiento and Camarero 2019; Wood et al. 2023). While accurate protein splicing typically dominates for most inteins, off-pathway reactions are possible. For example, N-terminal cleavage (NTC) can occur when the (thio) ester formed between the N-extein and intein is cleaved by an external nucleophile prior to ligation to the C-extein (FIG. 1B) (Mills et al. 2014). Several characterized mutations near the intein/extein junctions, as well as solution condition, can favor these cleavage reactions (Mills et al. 2014). Inteins with cleavage, but not splicing activities, have been particularly valuable for protein purification strategies. In one widely used intein-based purification technology (Wood et al. 1999; Prabhala et al. 2022), a target protein serves as one extein while an affinity tag serves as the other extein. Modified intein activity-resulting from mutations that block splicing but accelerate N-terminal cleavage-releases the purified target from the affinity tag at the end of the affinity purification process. Thus, intein-based protein purification systems provide convenient affinity tag/intein removal from the final target protein without the need for exogenous proteases.

Although intein C-terminal cleaving is spontaneous and can be made pH or temperature sensitive, conventional NTC systems require either extended incubation with high concentrations of an external nucleophile (e.g. Dithiothreitol; DTT) or high temperature (greater than 50° C.) (Mills et al. 2006; Amitai et al. 2009). While these systems have proven exceptionally useful, the conditions necessary to drive efficient cleavage (e.g. large temperature increases) often disrupt the folding of target proteins. Furthermore, the addition of an external nucleophile (e.g. DTT) at high concentrations can be prohibitively expensive for large-scale applications and is expected to disrupt disulfide bonds in proteins that possess them. Therefore, there is a need for inteins with improved abilities to perform controllable splicing and NTC under conditions suitable for large-scale applications.

### SUMMARY

The present disclosure provides polypeptides having intein activity (e.g., variants of the Thermococcus kodakarensis RadA intein) that efficiently perform controllable protein splicing and NTC at modest temperatures in the absence of DTT.

In an aspect, disclosed is a polypeptide having intein activity, comprising an amino acid sequence having at least about 80%, at least about 85%, at least about 90%, at least about 91%, at least about 92%, at least about 93%, at least about 94%, at least about 95%, at least about 96%, at least about 97%, at least about 98%, or at least about 99%, but less than 100%, sequence identity to the amino acid sequence of SEQ ID NO: 1 (TKΔE) or SEQ ID NO: 2 (TkPl), wherein the polypeptide has intein activity.

In some aspects, the polypeptide comprises an A residue at a position corresponding to position 1 and/or a position corresponding to position 172 of SEQ ID NO: 1 or SEQ ID NO: 2. In other aspects, the polypeptide comprises an A residue at position 1 and/or at position 172 of SEQ ID NO: 1 or SEQ ID NO: 2. In yet other aspects, the polypeptide comprises an A residue at a position corresponding to position 1 and/or a position corresponding to position 172 of SEQ ID NO: 1 or SEQ ID NO: 2. In still other aspects, the polypeptide comprises an A residue at position 1 and/or at position 172 of SEQ ID NO: 1 or SEQ ID NO: 2. In yet still other aspects, the polypeptide comprises a D residue at a position corresponding to position 67 and/or an A residue at a position corresponding to position 73 of SEQ ID NO: 1 or SEQ ID NO: 2. In other aspects, the polypeptide comprises a D residue at a position 67 and/or an A residue at position 73 of SEQ ID NO: 1 or SEQ ID NO: 2. In still other aspects, the polypeptide comprises a H residue at a position corresponding to position 67 and/or an A residue at a position corresponding to position 73 of SEQ ID NO: 1 or SEQ ID NO: 2. In further aspects, the polypeptide comprises a H residue at a position 67 and/or an A residue at position 73 of SEQ ID NO: 1 or SEQ ID NO: 2. In still further aspects, the polypeptide comprises an A residue, a D residue, an A residue and/or an A residue at positions corresponding to positions 1, 67, 73 and/or 172 of SEQ ID NO: 1 or SEQ ID NO: 2. In yet other aspects, the polypeptide comprises an A residue, a H residue, an A residue and/or an A residue at positions corresponding to positions 1, 67, 73 and/or 172 of SEQ ID NO: 1 or SEQ ID NO: 2. In certain aspects, the polypeptide of any one of clauses 1-9, further comprising an A residue, a D residue, an A residue and/or an A residue at positions 1, 67, 73 and/or 172 of SEQ ID NO: 1 or SEQ ID NO: 2. In other aspects, the polypeptide comprises an A residue, a H residue, an A residue and/or an A residue at positions 1, 67, 73 and/or 172 of SEQ ID NO: 1 or SEQ ID NO: 2.

In some aspects, the polypeptide has at least 90%, but less than 100%, sequence identity to the amino acid sequence of SEQ ID NO: 1 or SEQ ID NO: 2. In other aspects, the polypeptide has at least 95%, but less than 100%, sequence identity to the amino acid sequence of SEQ ID NO: 1 or SEQ ID NO: 2.

In some aspects, the polypeptide comprises, consists essentially of, or consists of: (a) the amino acid sequence of SEQ ID NO: 1 with an A residue at positions 1 and/or 172; and (b) the amino acid sequence of SEQ ID NO: 2 with an A residue at positions 1 and/or 172. In other aspects, the polypeptide of any one of clauses 1-21, wherein the polypeptide comprises, consists essentially of, or consists of: (a) the amino acid sequence of SEQ ID NO: 1 with an A residue at positions 1, a D or an H residue at position 67, an A residue at position 73, and/or an A residue at position 172; (b) the amino acid sequence of SEQ ID NO: 2 with an A residue at positions 1, a D or an H residue at position 67, an A residue at position 73, and/or an A residue at position 172.

In some aspects, the polypeptide exhibits impaired splicing and N-terminal cleavage (NTC) at temperatures below 20° C.; and/or the polypeptide exhibits controllable splicing and N-terminal cleavage at temperatures ranging from about 20° C. to about 50° C.

In another aspect, disclosed is a fusion protein comprising from N- to C-terminus an N-terminal extein polypeptide, an intein polypeptide, and a C-terminal extein polypeptide, wherein the intein polypeptide comprises a polypeptide having intein activity of the present disclosure.

In some aspects, the residue at the −1 position of the N-terminal extein polypeptide is selected to control the rate of NTC, wherein: (a) the −1 position of the N-terminal extein polypeptide is selected from a D, H, or K residue for fast NTC wherein at least 50% of the NTC occurs at 37° C. within about 2 hours; (b) the −1 position of the N-terminal extein polypeptide is selected from a E, F, G, L, M, Q, R, W or Y residue for moderate NTC wherein at least 50% of the NTC occurs at 37° C. within about 6 hours; or (c) the −1 position of the N-terminal extein polypeptide is selected from a A, C, I, N, P, S, T, or V residue for slow NTC wherein less than about 50% of the NTC occurs at 37° C. after about 6 hours.

In some aspects, the residue at the −1 position of the N-terminal extein polypeptide is a D residue.

In some aspects, the N-terminal extein polypeptide comprises a protein of interest.

In some aspects, the N-terminal extein polypeptide comprises an N-terminal affinity tag.

In some aspects, the C-terminal extein polypeptide comprises a reporter polypeptide.

In yet another aspect, disclosed is an intein-based protein expression and purification system, comprising: (a) the polypeptide having intein activity of the present disclosure or a fusion protein of the present disclosure; (b) a host cell comprising an expression vector or construct encoding the polypeptide or the fusion protein for expression of a protein of interest; and (c) a chromatography resin for purification of the protein of interest.

In some aspects, the expression vector or construct is operably linked to an inducible promoter.

In some aspects, the system comprises an inducer for promoting expression of the protein of interest.

In some aspects, the chromatography resin comprises metal affinity chromatography beads and the N-terminal affinity tag comprises a His-tag.

In some aspects, the system comprises a nucleophile to increase the rate of NTC.

In yet another aspect, disclosed is a kit comprising a polypeptide having intein activity of the present disclosure, a fusion protein of the present disclosure, or a system of the present disclosure, and instructions for using the polypeptide, the fusion protein, or the system for intein-based purification of a protein of interest.

In still yet another aspect, disclosed is a process for separating of a protein of interest on a chromatography resin, comprising: (a) contacting a fusion protein of the present disclosure optionally expressed using a system of the present disclosure with a chromatography resin to produce a chromatography resin-bound fusion protein, wherein contacting is performed at temperatures below 20° C. reduce or prevent NTC of the N-terminal extein polypeptide while the fusion protein remains bound to the chromatography resin; (b) incubating the chromatography resin-bound fusion protein at temperature greater than or equal to 20° C. to initiate NTC of the N-terminal extein polypeptide and release the polypeptide having intein activity and C-terminal extein polypeptide so that the N-terminal extein polypeptide remains bound to the chromatography resin; and (c) recovering the N-terminal extein polypeptide from the chromatography resin.

### BRIEF DESCRIPTION OF THE FIGURES

The patent or application file contains drawings executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1A and FIG. 1B show the mechanism of protein splicing and N-terminal cleavage. FIG. 1A shows splicing and FIG. 1B shows NTC schemes with −1, 1, and +1 positions, exteins (black), and intein (gray) indicated. Relevant chemical groups essential to protein splicing and NTC are drawn.

FIG. 2A, FIG. 2B, FIG. FIG. 2C, FIG. 2D, FIG. 2E and FIG. 2F show TkDE splicing and TkDE-AA N-terminal cleavage (NTC) is activated by increasing temperature. FIG. 1A shows a schematic of MBP-Intein-GFP (MIG) reporter. MBP is colored gray, intein is colored red, and GFP is colored green. GFP-containing fluorescent products are visualized in-gel following semi-native PAGE. FIG. 2B shows splicing of TkDE in MIG reporter at different temperatures for 60 minutes. Precursor (M-I-G) and ligated exteins (M-G) bands are indicated. FIG. 2C shows quantification of splicing at different temperatures from three independent splicing reactions. FIG. 2D shows a MIG reporter with mutations to allow for NTC rather than splicing. Colored as in panel A. FIG. 2E shows NTC TkDE-AA in MIG reporter over 180 minutes at different temperatures. Precursor (M-I-G) and intein-GFP (I-G) products are indicated. FIG. 2F shows Quantification of splicing at different temperatures from three independent cleavage reactions. Error bars indicate standard deviation. When error bars are not shown in FIG. 2C and FIG. 2F, they are smaller than the symbol.

FIG. 3A, FIG. 3B, and FIG. 3C show TkDE-AA NTC with 20 common amino acids in the −1 position. FIG. 2A shows the quantification of NTC with different residues at −1 position of TkDE-AA after incubation for 2 (white bars), 6 (gray bars), and 20 (black bars) hours at 37° C. The relative rates of NTC for each −1 position are indicated with red (fast), green (moderate), or blue (slow). One letter amino acids abbreviations are shown. FIG. 3B shows examples of relative NTC rates for a fast, moderate, and slow −1 residue. NTC after 2 hours at 37° C. for TkDE-AA with -1H (fast), -1M (moderate), and -1I (slow). FIG. 3C shows TkDE-AA-1D (Asp in −1 position) does not undergo premature NTC following expression at 15° C. for 20 hours and proceeds following incubation at 37° C. for indicated times. Quantification in FIG. 3A is done as described in FIG. 1A and FIG. 1B.

FIG. 4A, FIG. 4B, FIG. 4C, and FIG. 4D show homologous intein from P. horikoshii is not prone to NTC and on-column NTC of the TkPl-AA variant. FIG. 4A shows TkPl-AA, but not Ph-AA, in the MIG reporter undergoes efficient NTC during incubation at 37° C. for 16 hours. FIG. 4B shows quantification of NTC for TkPl-AA and Ph-AA following incubation at 37° C. for 16 hours. FIG. 4C shows TkPl-AA MIG precursor intein can be purified, and, after incubation for 16 hours at 37° C., two products are observed by Coomassie staining with sizes consistent with the cleaved MBP and intein-GFP. FIG. 4D shows TkPl-AA MIG precursor intein maintains NTC activity at 37° C. while on beads following affinity resin capture and washing. In FIG. 4D, T indicates the product eluted from total amount of beads prior to incubation at 37° C., R is the product released into solution following incubation at 37° C., and U is the product that remained on the beads following incubation at 37° C. In FIG. 4A, FIG. 4B, FIG. 4C and FIG. 4D, TO is as described in FIG. 1A and FIG. 1B shows and precursor (M-I-G), MBP (M) and intein-GFP (I-G) bands are indicated. Quantification and error are determined based on at least three independent NTC reactions as described in FIG. 1A and FIG. 1B.

FIG. 5 shows a cartoon of intein-based N-terminal cleavage protein purification strategy using an intein with modified activity resulting from mutations that block splicing but accelerate N-terminal cleavage releasing a purified target from an affinity tag at the end of an affinity purification process. The protein of interest (POI) to be purified is shown in blue as the N-extein. The intein (INT) and affinity tag (AT) are shown in pink. In step 1, the POI-INT-AT is isolated from impurities using chromatography. In step 2, the POI is released from the INT-AT in a purified form by incubation at 37° C.

FIG. 6A, FIG. 6B, and FIG. 6C show zinc blocks TkDE splicing and TkDE-AA N-terminal cleavage (NTC). FIG. 6A shows splicing of TkDE and FIG. 6B shows NTC of TkDE-AA are blocked by 10 mM zinc. In FIG. 6A and FIG. 6B, samples were incubated at 37° C. for the indicated times, and precursor (M-I-G), ligated exteins (M-G), and intein-GFP (I-G) bands are labeled. FIG. 6C shows quantification of TkDE splicing and TkDE-AA NTC in the presence or absence of zinc in FIG. 6A and FIG. 6B as described in FIG. 1A and FIG. 1B. When error bars are not shown in FIG. 6C, they are smaller than the symbol.

FIG. 7A, FIG. 7B, and FIG. 7C show hydroxylamine (HA), but not dithiothreitol (DTT), accelerates TkDE-AA NTC (NTC), and the homing endonuclease does not prevent NTC. FIG. 7A shows TkDE-AA NTC at 37° C. in the absence of an external nucleophile (water) or presence of 50 mM DTT or HA. Precursor (M-I-G) and intein-GFP (I-G) bands are indicated. FIG. 7B shows quantification of TkDE-AA NTC in the presence of water, DTT, or HA at indicated times as described in FIG. 1A and FIG. 1B. FIG. 7C shows quantification of TkDE-AA and TkE-AA NTC following expression in E. coli at 15° C. for ˜20 hours. When error bars are not shown in FIG. 7B and FIG. 7C, they are smaller than the symbol or line.

FIG. 8 shows a clustal alignment of the TkDE (SEQ ID NO: 1) and Ph (SEQ ID NO: 4) and RadA inteins. The red box indicates the residues from the Ph intein exchanged with the TkDE intein to form the TkPl intein. The site of homing endonuclease deletion within the TkDE intein is underlined.

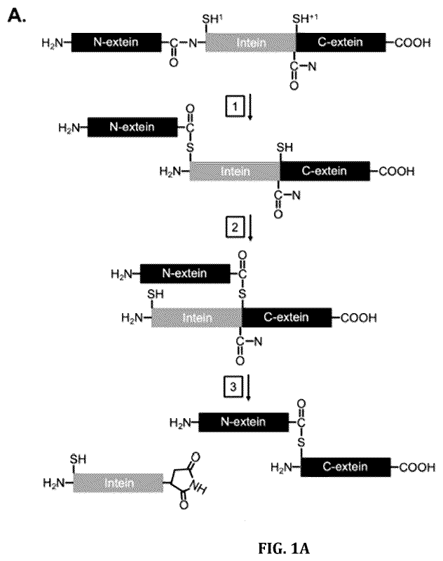

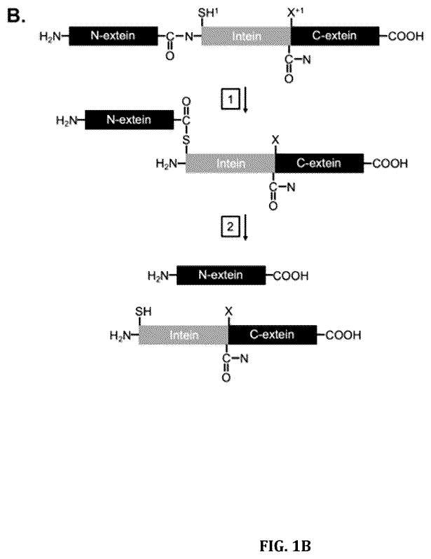

### DETAILED DESCRIPTION OF THE INVENTION

Conditional protein splicing, wherein the rate and accuracy of protein splicing is dependent on an external signal, naturally occurs for several inteins and has even been engineered through directed evolution (Buskirk et al. 2004; Skretas and Wood 2005; Lennon and Belfort 2017). While temperature has previously been used as an effective signal to stimulate splicing and NTC (Mills et al. 2006), the present disclosure provides polypeptides having intein activity (e.g., variants of the T. kodakarensis RadA intein) that unexpectedly perform controllable splicing and NTC activity at lower temperatures. The examples herein demonstrate that intein activity is largely blocked during expression at 15° C. in E. coli, but that the intein can efficiently undergo splicing and NTC at temperatures greater than 20° C. In another intein-based, temperature-dependent system for NTC, incubation for 5 hours at 55° C. was needed to stimulate approximately 70% NTC (Mills et al. 2006). It is of note that this previous system did not require the addition of an external nucleophile (e.g. DTT). This is comparable to the level of NTC from an exemplary polypeptide having intein activity of the present disclosure (e.g., TkDE-AA) incubated for the same amount of time at just 21° C. Therefore, compared to this previously reported system, NTC by the polypeptides of the present disclosure unexpectedly occurs at a substantially lower temperature.

The examples of the present disclosure also unexpectedly demonstrate that exemplary polypeptides having intein-activity of the present disclosure (e.g., TkDE-AA and TkPl-AA) do not require an external nucleophile to stimulate NTC. Interestingly, while DTT does not accelerate NTC by an exemplary polypeptide having intein activity of the present disclosure (e.g., TkDE-AA), the smaller nucleophile HA increases the rate of NTC. These results suggest the scissile bond between the N-extein and intein is in an unusual conformation compared to other inteins as it is accessible to HA, but not to the larger DTT.

TkDE-AA can accommodate all 20 common amino acids in the −1 position, with the rate of NTC varying depending on residue identity. Importantly, all undergo near complete NTC after incubation at 37° C. for 20 hours. This suggests that this intein variant could accommodate any C-terminal residue of a protein of interest (POI) to be purified. Surprisingly, it was observed that aspartate can be accommodated in the −1 position of TkDE-AA without premature NTC. This appears to solve the limitation of previous intein-based systems, expanding potential intein uses for proteins with a C-terminal aspartate.

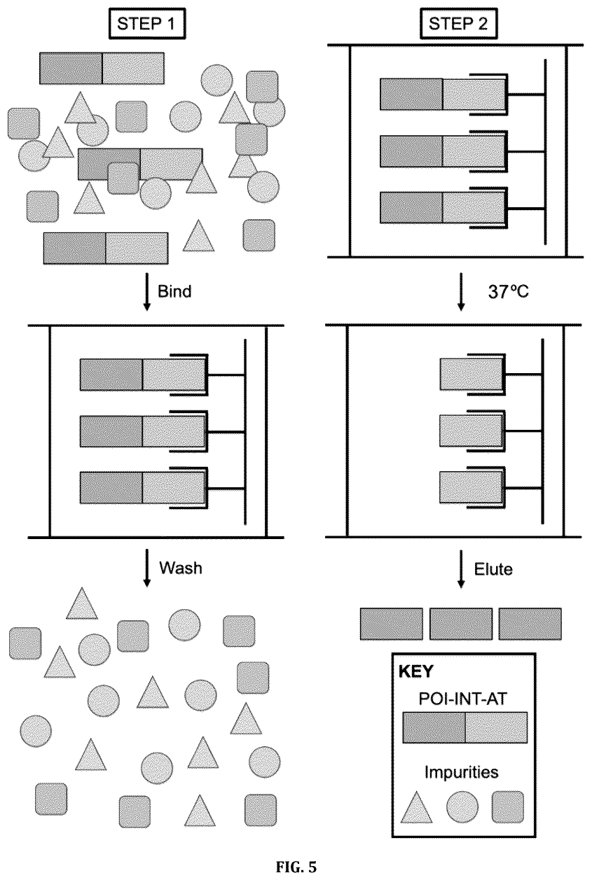

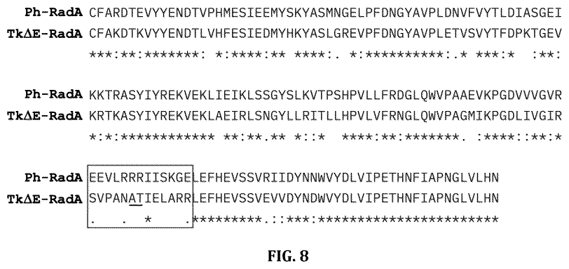

The examples of the present disclosure show that the RadA mini-intein from P. horikoshii, which is homologous to the T. kodakarensis RadA intein except that it naturally lacks a HEN domain (FIG. 8), is not prone to NTC under conditions where TkPl-AA cleavage is efficient. Finally, the potential of the TkPl-AA intein is demonstrated, a fusion between TkDE-AA and the Ph intein, to efficiently undergo NTC while bound to affinity resin at 37° C. These results suggest that the TkPl-AA intein may be useful for intein-based purification systems at moderate temperatures in the absence of an external nucleophile. In this proposed purification scheme, the TkPl-AA intein and affinity tag (AT) would be fused to the C-terminus of a POI to be purified. Following overexpression at 15° C., the POI-intein-AT precursor protein would be bound to chromatography resin via the AT at temperatures below 20° C. After washing away impurities, TkPl-AA-mediated NTC would be induced by temperature at or above 37° C., resulting in the elution of the pure, tagless POI (FIG. 5).

The polypeptides having intein-activity of the present disclosure (e.g., TkDE, TkDE-AA, and TkPl-AA) display a unique range of temperature-activated splicing and NTC. This relatively fast splicing and NTC at 37° C. and comparatively low activity during expression at 15° C. represents both a temperature range and time scale that could prove useful for many organisms of interest to the research community.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, an “affinity tag” refers to a peptide sequence that is fused to a target protein to facilitate its purification from a complex mixture (e.g., purification of an N-terminal extein protein of interest using an intein-based purification system of the present disclosure). The affinity tag is designed to bind with high specificity to a corresponding ligand or binding partner immobilized on a solid support, such as a column or resin, allowing for the isolation of the target protein through affinity chromatography. Exemplary affinity tags include, without limitation, polyhistidine (His-tag), glutathione S-transferase (GST), and maltose-binding protein (MBP), which can be employed in conjunction with a variant intein polypeptide of the present disclosure to enable controlled cleavage and release of the purified target protein from the affinity matrix. The use of an affinity tag in an intein-based purification system enhances the efficiency and purity of the target protein by simplifying the purification process, allowing for easier recovery and minimizing non-specific interactions with other proteins in the sample.

As used herein, “controllable NTC” refers to refers to the ability to selectively remove the N-terminal extein of a fusion protein comprising a variant polypeptide having intein activity of the present disclosure under predetermined conditions. This process enables the precise regulation of the timing and extent of cleavage to facilitate applications such as protein purification, functionalization, or the generation of protein fragments. Controllable N-terminal cleavage can be achieved by employing specific chemical reagents, temperature changes, or other external stimuli that trigger the cleavage reaction at the N-terminus of the polypeptide.

As used herein, “controllable splicing” refers to the regulated process by which an intein variant polypeptide of the present disclosure facilitates the precise joining of two polypeptide segments through the removal of an intervening sequence, allowing for the formation of a contiguous protein. This process can be selectively triggered under specific conditions, such as the presence of chemical agents, changes in temperature, or alterations in pH, enabling the manipulation of protein constructs for various applications. Exemplary controllable splicing methods include, without limitation, inteins engineered to undergo splicing in response to specific small molecules, light activation, or other external stimuli that induce the excision of the intein and ligation of the flanking protein segments. This capability allows for the controlled assembly of proteins in synthetic biology, protein engineering, and therapeutic applications, enabling the generation of functional protein variants with tailored properties and activities.

As used herein, “impaired NTC” refers to a reduction or loss of the ability to effectively cleave the N-terminal portion of a polypeptide or protein, for example, an N-terminal extein of a fusion protein comprising a variant polypeptide having intein activity of the present disclosure. In some instances, a variant polypeptide having intein activity of the present disclosure exhibits a reduction of NTC activity, for example, by at least 5%, at least 10%, at least 15%, at least 20%, at least 30%, at least 35%, at least 40%, at least 45%, at least 50%, at least 55%, a least 60%, at least 65%, at least 70%, at least 75%, at least 80%, at least 85%, at least 95%, at least 96%, at least 97%, at least 98%, or at least 99%. In other instances, a variant polypeptide having intein activity of the present disclosure exhibits complete loss of NTC activity. This impairment may result from specific mutations, structural modifications, or environmental conditions that adversely affect the cleavage mechanism, leading to incomplete or inefficient removal of the N-terminal segment. Exemplary instances of impaired N-terminal cleavage include, without limitation, intein variants that possess amino acid substitutions within the cleavage site that hinder the catalytic activity required for N-terminal processing, or those whose activity is diminished under certain conditions, such as suboptimal pH or temperature. Impaired N-terminal cleavage can impact the functionality of the resulting polypeptide, affecting its stability, activity, or interaction with other biomolecules, and can be used in the design of intein-based systems, for example, for controlled protein processing (e.g., expression and purification).

As used herein, “impaired splicing” refers to a reduced efficiency or complete failure of the splicing process mediated by an intein variant polypeptide of the present disclosure, which results in the inability to effectively join two polypeptide segments (e.g., N-terminal extein and C-terminal extein) due to the incomplete or improper excision of the intervening intein sequence. In some instances, a variant polypeptide having intein activity of the present disclosure exhibits a reduced efficiency of splicing activity, for example, by at least 5%, at least 10%, at least 15%, at least 20%, at least 30%, at least 35%, at least 40%, at least 45%, at least 50%, at least 55%, a least 60%, at least 65%, at least 70%, at least 75%, at least 80%, at least 85%, at least 95%, at least 96%, at least 97%, at least 98%, or at least 99%. In other instances, a variant polypeptide having intein activity of the present disclosure exhibits complete loss of splicing activity. This impairment may arise from specific mutations, structural alterations, or unfavorable environmental conditions that disrupt the normal splicing mechanism critical for the formation of a contiguous protein. Exemplary instances of impaired splicing include, without limitation, intein variant polypeptides that exhibit mutations at key residues essential for the splicing reaction, leading to reduced catalytic activity, or those that are sensitive to temperature, pH, or the presence of specific chemical inducers that inhibit the splicing process. Impaired splicing can affect the overall functionality of the resultant protein, influencing its stability, activity, or interactions with other biomolecules, and may be utilized in applications involving controlled protein assembly or in the investigation of splicing mechanisms in protein engineering and synthetic biology.

As used herein, “Tk” is14 an abbreviation for Thermococcus kodakarensis. As used herein, “TkDE” and “TkDE” are used interchangeably to refer to a variant of the Tk radA intein polypeptide in which the homing endonuclease domain (HED) consisting of residues 276-585 of the wild-type Tk radA intein amino acid sequence were deleted. This exemplary intein variant polypeptide of the present disclosure has the amino acid sequence of SEQ ID NO: 1. As used herein, “TkPl” refers to a variant of the TkDE polypeptide in which amino acid residues flanking the deleted HED of the Tk radA intein were replaced with a stretch of amino acids from the Pyrococcus horikoshii radA intein. The amino acid sequence of the exemplary TkPl variant intein polypeptide of the present disclosure is set forth in SEQ ID NO: 2. The amino acid sequence of the wild-type Tk radA intein polypeptide sequence is set forth in SEQ ID NO: 3.

As used herein, “Ph” is an abbreviation for Pyrococcus horikoshii. The wild-type amino acid sequence of the Ph radA intein polypeptide is set forth in SEQ ID NO: 4. Amino acid residues 120 to 133 of exemplary intein variant polypeptide TkDE of SEQ ID NO: 1 were replaced with amino acid residues 120 to 133 of SEQ ID NO: 4 to produce the exemplary intein variant polypeptide TkPl of SEQ ID NO: 2.

SEQ ID NOS. 1-4 are shown in the table below:

| SEQ ID | |
|---|---|
| NO: | SEQUENCE |
| 1 | CFAKDTKVYYENDTLVHFESIEDMYHKYASLGREVPFDNG |
| | YAVPLETVSVYTFDPKTGEVKRTKASYIYREKVEKLAEIR |
| | LSNGYLLRITLLHPVLVFRNGLQWVPAGMIKPGDLIVGIR |
| | SVPANATIELARRLEFHEVSSVEVVDYNDWVYDLVIPETH |
| | NFIAPNGLVLHN |
| 2 | CFAKDTKVYYENDTLVHFESIEDMYHKYASLGREVPFDNG |
| | YAVPLETVSVYTFDPKTGEVKRTKASYIYREKVEKLAEIR |
| | LSNGYLLRITLLHPVLVFRNGLQWVPAGMIKPGDLIVGIR |
| | EEVLRRRIISKGELEFHEVSSVEVVDYNDWVYDLVIPETH |
| | NFIAPNGLVLHN |
| 3 | CFAKDTKVYYENDTLVHFESIEDMYHKYASLGREVPFDNG |
| | YAVPLETVSVYTFDPKTGEVKRTKASYIYREKVEKLAEIR |
| | LSNGYLLRITLLHPVLVFRNGLQWVPAGMIKPGDLIVGIR |
| | SVPANAATIEESEAYFLGLFVAEGTSNPLSITTGSEELKD |
| | EFIVSFIEDHDGYTPTVEVRRGLYRILFRKKTAEWLGELA |
| | TSNASTKVVPERVLNAGESAIAAFLAGYLDGDGYLTESIV |
| | ELVTKSRELADGLVFLLKRLGITPRISQKTIEGSVYYRIY |
| | ITGEDRKTFEKVLEKSRIKPGMNEGGVGRYPPALGKFLGK |
| | LYSEFRLPKRDNETAYHILTRSRNVWFTEKTLSRIEEYFR |
| | EALEKLSEARKALEMGDKPELPFPWTAITKYGFTDRQVAN |
| | YRTRGLPKRPELKEKVVSALLKEIERLEGVAKLALETIEL |
| | ARRLEFHEVSSVEVVDYNDWVYDLVIPETHNFIAPNGLVL |
| | HN |
| 4 | CFARDTEVYYENDTVPHMESIEEMYSKYASMNGELPFDNG |
| | YAVPLDNVFVYTLDIASGEIKKTRASYIYREKVEKLIEIK |
| | LSSGYSLKVTPSHPVLLFRDGLQWVPAAEVKPGDVVVGVR |
| | EEVLRRRIISKGELEFHEVSSVRIIDYNNWVYDLVIPETH |
| | NFIAPNGLVLHN |

The present disclosure refers to various amino acid residues by their full name and by their requisite one and three letter codes as shown in the table below:

| | Three | One |
|---|---|---|
| Amino acid Residue Full Name | letter code | letter code |
| Alanine | ala | A |
| Arginine | arg | R |
| Asparagine | asn | N |
| aspartic acid | asp | D |
| asparagine or aspartic acid | asx | B |
| Cysteine | cys | C |
| glutamic acid | glu | E |
| Glutamine | gln | Q |
| glutamine or glutamic acid | glx | Z |
| Glycine | gly | G |
| Histidine | his | H |
| Isoleucine | ile | I |
| Leucine | leu | L |
| Lysine | lys | K |
| Methionine | met | M |
| Phenylalanine | phe | F |
| Proline | pro | P |
| Serine | ser | S |
| Threonine | thr | T |
| Tryptophan | trp | W |
| Tyrosine | tyr | Y |
| Valine | val | V |

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### Polypeptides

Aspects of the disclosure relate to variant polypeptides having intein activity, for example, polypeptides exhibiting improved controllable splicing and NTC compared conventional wild type intein polypeptides. The variant polypeptides of the present disclosure can be used in intein-based protein expression and purification systems, for example, for temperature controllable NTC while a protein of interest is bound to an affinity chromatography resin to release the intein and C-terminal extein to facilitate purification and separation of the protein of interest.

In an aspect, the present disclosure provides a variant polypeptide having intein activity, comprising an amino acid sequence having at least about 80%, at least about 85%, at least about 90%, at least about 91%, at least about 92%, at least about 93%, at least about 94%, at least about 95%, at least about 96%, at least about 97%, at least about 98%, or at least about 99%, but less than 100%, sequence identity to the amino acid sequence of SEQ ID NO: 1, wherein the polypeptide has intein activity.

In another aspect, the present disclosure provides a variant polypeptide having intein activity, comprising an amino acid sequence having at least about 80%, at least about 85%, at least about 90%, at least about 91%, at least about 92%, at least about 93%, at least about 94%, at least about 95%, at least about 96%, at least about 97%, at least about 98%, or at least about 99%, but less than 100%, sequence identity to the amino acid sequence of SEQ ID NO: 2, wherein the polypeptide has intein activity.

In some aspects, the polypeptide comprises an alanine (A) residue at a position corresponding to position 1 of SEQ ID NO: 1. In other aspects, the polypeptide comprises an A residue at a position corresponding to position 1 of SEQ ID NO: 2. In yet other aspects, the polypeptide comprises an A residue at a position corresponding to position 172 of SEQ ID NO: 1. In still other aspects, the polypeptide comprises an A residue at a position corresponding to position 172 of SEQ ID NO: 2. In further aspects, the polypeptide comprises an A residue at positions corresponding to positions 1 and 172 of SEQ ID NO: 1. In still other aspects, the polypeptide comprises an A residue at positions corresponding to positions 1 and 172 of SEQ ID NO: 2.

In some aspects, the polypeptide comprises an A residue at position 1 of SEQ ID NO: 1. In other aspects, the polypeptide comprises an A residue at position 1 of SEQ ID NO: 2. In yet other aspects, the polypeptide comprises an A residue at position 172 of SEQ ID NO: 1. In still other aspects, the polypeptide comprises an A residue at position 172 of SEQ ID NO: 2. In further aspects, the polypeptide comprises an A residue at positions 1 and 172 of SEQ ID NO: 1. In still other aspects, the polypeptide comprises an A residue at positions 1 and 172 of SEQ ID NO: 2.

In some aspects, the polypeptide comprises an aspartic acid (D) residue at a position corresponding to position 67 of SEQ ID NO: 1. In some aspects, the polypeptide comprises a histidine (H) residue at a position corresponding to position 67 of SEQ ID NO:

1. In other aspects, the polypeptide comprises a D residue at a position corresponding to position 67 of SEQ ID NO: 2. In other aspects, the polypeptide comprises an H residue at a position corresponding to position 67 of SEQ ID NO: 2.

In yet other aspects, the polypeptide comprises an A residue at a position corresponding to position 73 of SEQ ID NO: 1. In still other aspects, the polypeptide comprises an A residue at a position corresponding to position 73 of SEQ ID NO: 2.

In further aspects, the polypeptide comprises a D residue and an A residue at positions corresponding to positions 67 and 73 of SEQ ID NO: 1. In further aspects, the polypeptide comprises a H residue and an A residue at positions corresponding to positions 67 and 73 of SEQ ID NO: 1. In further aspects, the polypeptide comprises a D residue and an A residue at positions corresponding to positions 67 and 73 of SEQ ID NO: 2. In further aspects, the polypeptide comprises a H residue and an A residue at positions corresponding to positions 67 and 73 of SEQ ID NO: 2.

In some aspects, the polypeptide comprises an D residue at position 67 of SEQ ID NO: 1. In some aspects, the polypeptide comprises an H residue at position 67 of SEQ ID NO: 1. In other aspects, the polypeptide comprises an D residue at position 67 of SEQ ID NO: 2. In other aspects, the polypeptide comprises an H residue at position 67 of SEQ ID NO: 2. In yet other aspects, the polypeptide comprises an A residue at position 73 of SEQ ID NO: 1. In still other aspects, the polypeptide comprises an A residue at position 73 of SEQ ID NO: 2. In further aspects, the polypeptide comprises D and A residues at positions 67 and 73 of SEQ ID NO: 1. In further aspects, the polypeptide comprises H and A residues at positions 67 and 73 of SEQ ID NO: 1. In still other aspects, the polypeptide comprises D and A residues at positions 67 and 73 of SEQ ID NO: 2. In still other aspects, the polypeptide comprises H and A residues at positions 67 and 73 of SEQ ID NO: 2.

In some aspects, the polypeptide comprises an A residue and a D residue at positions corresponding to positions 1 and 67 of SEQ ID NO: 1. In some aspects, the polypeptide comprises an A residue and a H residue at a positions corresponding to positions 1 and 67 of SEQ ID NO: 1. In other aspects, the polypeptide comprises an A residue and a D residue at positions corresponding to positions 1 and 67 of SEQ ID NO: 2. In other aspects, the polypeptide comprises an A residue and an H residue at positions corresponding to positions 1 and 67 of SEQ ID NO: 2.

In yet other aspects, the polypeptide comprises A residues at positions corresponding to positions 1 and 73 of SEQ ID NO: 1. In still other aspects, the polypeptide comprises A residues at positions corresponding to positions 1 and 73 of SEQ ID NO: 2.

In further aspects, the polypeptide comprises an A residue, a D residue and an A residue at positions corresponding to positions 1, 67 and 73 of SEQ ID NO: 1. In further aspects, the polypeptide comprises an A residue, a H residue and an A residue at positions corresponding to positions 1, 67 and 73 of SEQ ID NO: 1. In further aspects, the polypeptide comprises an A residue, a D residue and an A residue at positions corresponding to positions 1, 67 and 73 of SEQ ID NO: 2. In further aspects, the polypeptide comprises an A residue, a H residue and an A residue at positions corresponding to positions 1, 67 and 73 of SEQ ID NO: 2.

In some aspects, the polypeptide comprises an A residue and a D residue at positions 1 and 67 of SEQ ID NO: 1. In some aspects, the polypeptide comprises an A residue and an H residue at position 1 and 67 of SEQ ID NO: 1. In other aspects, the polypeptide comprises an A residue and a D residue at positions 1 and 67 of SEQ ID NO: 2. In other aspects, the polypeptide comprises an A residue and a H residue at positions 1 and 67 of SEQ ID NO: 2. In yet other aspects, the polypeptide comprises A residues at positions 1 and 73 of SEQ ID NO: 1. In still other aspects, the polypeptide comprises an A residues at positions 1 and 73 of SEQ ID NO: 2.

In further aspects, the polypeptide comprises A, D and A residues at positions 1, 67 and 73 of SEQ ID NO: 1. In further aspects, the polypeptide comprises A, H and A residues at positions 1, 67 and 73 of SEQ ID NO: 1. In still other aspects, the polypeptide comprises A, D and A residues at positions 1, 67 and 73 of SEQ ID NO: 2. In still other aspects, the polypeptide comprises A, H and A residues at positions 1, 67 and 73 of SEQ ID NO: 2.

In some aspects, the polypeptide comprises a D residue and an A residue at position corresponding to positions 67 and 172 of SEQ ID NO: 1. In some aspects, the polypeptide comprises an H residue and an A residue at positions corresponding to positions 67 and 172 of SEQ ID NO: 1. In other aspects, the polypeptide comprises a D residue and an A residue at positions corresponding to positions 67 and 172 of SEQ ID NO: 2. In other aspects, the polypeptide comprises a H residue and an A residue at positions corresponding to positions 67 and 172 of SEQ ID NO: 2.

In yet other aspects, the polypeptide comprises an A residue at positions corresponding to positions 73 and 172 of SEQ ID NO: 1. In still other aspects, the polypeptide comprises an A residue at positions corresponding to positions 73 and 172 of SEQ ID NO: 2.

In further aspects, the polypeptide comprises a D residue, an A residue, and an A residue at positions corresponding to positions 67, 73 and 172 of SEQ ID NO: 1. In further aspects, the polypeptide comprises a H residue, an A residue, and an A residue at positions corresponding to positions 67, 73 and 172 of SEQ ID NO: 1. In further aspects, the polypeptide comprises a D residue, an A residue, and an A residue at positions corresponding to positions 67, 73 and 172 of SEQ ID NO: 2. In further aspects, the polypeptide comprises a H residue, an A residue and an A residue at positions corresponding to positions 67, 73 and 172 of SEQ ID NO: 2.

In some aspects, the polypeptide comprises a D residue and an A residue at position 67 and 172 of SEQ ID NO: 1. In some aspects, the polypeptide comprises an H and an A residue at positions 67 and 172 of SEQ ID NO: 1. In other aspects, the polypeptide comprises a D and an A residue at position 67 and 172 of SEQ ID NO: 2. In other aspects, the polypeptide comprises a H and an A residue at positions 67 and 172 of SEQ ID NO: 2. In yet other aspects, the polypeptide comprises an A residue at positions 73 and 172 of SEQ ID NO: 1. In still other aspects, the polypeptide comprises an A residue at positions 73 and 172 of SEQ ID NO: 2.

In further aspects, the polypeptide comprises D, A, and A residues at positions 67, 73 and 172 of SEQ ID NO: 1. In further aspects, the polypeptide comprises H, A and A residues at positions 67, 73 and 172 of SEQ ID NO: 1. In still other aspects, the polypeptide comprises D, A, and A residues at positions 67, 73 and 172 of SEQ ID NO: 2. In still other aspects, the polypeptide comprises H, A and A residues at positions 67, 73 and 172 of SEQ ID NO: 2.

In some aspects, the polypeptide comprises an A, D, and an A residue at positions corresponding to positions 1, 67 and 172 of SEQ ID NO: 1. In some aspects, the polypeptide comprises an A, H, and an A residue at positions corresponding to positions 1, 67 and 172 of SEQ ID NO: 1. In other aspects, the polypeptide comprises an A, D and an A residue at positions corresponding to positions 1, 67 and 172 of SEQ ID NO: 2. In other aspects, the polypeptide comprises an A, H, and an A residue at positions corresponding to positions 1, 67 and 172 of SEQ ID NO: 2.

In yet other aspects, the polypeptide comprises an A residue at positions corresponding to positions 1, 73 and 172 of SEQ ID NO: 1. In still other aspects, the polypeptide comprises an A residue at positions corresponding to positions 1, 73 and 172 of SEQ ID NO: 2.

In further aspects, the polypeptide comprises an A, D, A, and an A residue at positions corresponding to positions 1, 67, 73 and 172 of SEQ ID NO: 1. In further aspects, the polypeptide comprises an A, H, A, and an A residue at positions corresponding to positions 1, 67, 73 and 172 of SEQ ID NO: 1. In further aspects, the polypeptide comprises an A, D, A, and an A residue at positions corresponding to positions 1, 67, 73 and 172 of SEQ ID NO: 2. In further aspects, the polypeptide comprises an A, H, A, and an A residue at positions corresponding to positions 1, 67, 73 and 172 of SEQ ID NO: 2.

In some aspects, the polypeptide comprises an A, D, and an A residue at position 1, 67 and 172 of SEQ ID NO: 1. In some aspects, the polypeptide comprises an A, H and an A residue at positions 1, 67 and 172 of SEQ ID NO: 1. In other aspects, the polypeptide comprises an A, D and an A residue at positions 1, 67 and 172 of SEQ ID NO: 2. In other aspects, the polypeptide comprises an A, H and an A residue at positions 1, 67 and 172 of SEQ ID NO: 2.

In yet other aspects, the polypeptide comprises an A residue at positions 1, 73 and 172 of SEQ ID NO: 1. In still other aspects, the polypeptide comprises an A residue at positions 1, 73 and 172 of SEQ ID NO: 2.

In further aspects, the polypeptide comprises A, D, A, and A residues at positions 1, 67, 73 and 172 of SEQ ID NO: 1. In further aspects, the polypeptide comprises A, H, A and A residues at positions 1, 67, 73 and 172 of SEQ ID NO: 1. In still other aspects, the polypeptide comprises A, D, A, and A residues at positions 1, 67, 73 and 172 of SEQ ID NO: 2. In still other aspects, the polypeptide comprises A, H, A and A residues at positions 1, 67, 73 and 172 of SEQ ID NO: 2.

In some aspects, the variant polypeptides comprise an amino acid sequence having at least 90%, e.g., at least 91%, at least 92%, at least 93%, at least 94%, at least 95%, at least 96%, at least 97%, at least 98%, at least 99%, but less than 100%, sequence identity to the amino acid sequence of SEQ ID NO: 1. In other aspects, the variant polypeptides comprise an amino acid sequence having at least 90%, e.g., at least 91%, at least 92%, at least 93%, at least 94%, at least 95%, at least 96%, at least 97%, at least 98%, at least 99%, but less than 100%, sequence identity to the amino acid sequence of SEQ ID NO: 2.

In an embodiment, the variant polypeptide has at least 91%, but less than 100%, sequence identity to the amino acid sequence of SEQ ID NO: 1. In another embodiment, the variant polypeptide has at least 92%, but less than 100%, sequence identity to the amino acid sequence of SEQ ID NO: 1. In another embodiment, the variant polypeptide has at least 93%, but less than 100%, sequence identity to the amino acid sequence of SEQ ID NO: 1. In an embodiment, the variant polypeptide has at least 94%, but less than 100%, sequence identity to the amino acid sequence of SEQ ID NO: 1. In another embodiment, the variant polypeptide has at least 95%, but less than 100%, sequence identity to the amino acid sequence of SEQ ID NO: 1. In another embodiment, the variant polypeptide has at least 96%, but less than 100%, sequence identity to the amino acid sequence of SEQ ID NO: 1. In another embodiment, the variant polypeptide has at least 97%, but less than 100%, sequence identity to the amino acid sequence of SEQ ID NO: 1. In another embodiment, the variant polypeptide has at least 98%, but less than 100%, sequence identity to the amino acid sequence of SEQ ID NO: 1. In another embodiment, the variant polypeptide has at least 99%, but less than 100%, sequence identity to the amino acid sequence of SEQ ID NO: 1.

In an embodiment, the variant polypeptide has at least 91%, but less than 100%, sequence identity to the amino acid sequence of SEQ ID NO: 2. In another embodiment, the variant polypeptide has at least 92%, but less than 100%, sequence identity to the amino acid sequence of SEQ ID NO: 2. In another embodiment, the variant polypeptide has at least 93%, but less than 100%, sequence identity to the amino acid sequence of SEQ ID NO: 2. In an embodiment, the variant polypeptide has at least 94%, but less than 100%, sequence identity to the amino acid sequence of SEQ ID NO: 2. In another embodiment, the variant polypeptide has at least 95%, but less than 100%, sequence identity to the amino acid sequence of SEQ ID NO: 2. In another embodiment, the variant polypeptide has at least 96%, but less than 100%, sequence identity to the amino acid sequence of SEQ ID NO: 2. In another embodiment, the variant polypeptide has at least 97%, but less than 100%, sequence identity to the amino acid sequence of SEQ ID NO: 2. In another embodiment, the variant polypeptide has at least 98%, but less than 100%, sequence identity to the amino acid sequence of SEQ ID NO: 1. In another embodiment, the variant polypeptide has at least 99%, but less than 100%, sequence identity to the amino acid sequence of SEQ ID NO: 2.

In some aspects, the variant polypeptide comprises, consists essentially of, or consists of the amino acid sequence of SEQ ID NO: 1 with an A residue at position 1. In other aspects, the variant polypeptide comprises, consists essentially of, or consists of the amino acid sequence of SEQ ID NO: 1 with an A residue at position 172. In yet other aspects, the variant polypeptide comprises, consists essentially of, or consists of the amino acid sequence of SEQ ID NO: 1 with an A residue at position 1 and 172. In other aspects, the variant polypeptide comprises, consists essentially of, or consists of the amino acid sequence of SEQ ID NO: 1 with a D residue at position 67. In yet other aspects, the variant polypeptide comprises, consists essentially of, or consists of the amino acid sequence of SEQ ID NO: 1 with a H residue at position 67. In still other aspects, the variant polypeptide comprises, consists essentially of, or consists of the amino acid sequence of SEQ ID NO: 1 with an A residue at position 73. In certain aspects, the variant polypeptide comprises, consists essentially of, or consists of the amino acid sequence of SEQ ID NO: 1 with a D residue at position 67 and an A residue at position 73. In yet other aspects, the variant polypeptide comprises, consists essentially of, or consists of the amino acid sequence of SEQ ID NO: 1 with a H residue at position 67 and an A residue at position 73. In still other aspects, the variant polypeptide comprises, consists essentially of, or consists of the amino acid sequence of SEQ ID NO: 1 with an A residue at position 1 and a D residue at position 67. In still other aspects, the variant polypeptide comprises, consists essentially of, or consists of the amino acid sequence of SEQ ID NO: 1 with an A residue at position 1 and a H residue at position 67. In still other aspects, the variant polypeptide comprises, consists essentially of, or consists of the amino acid sequence of SEQ ID NO: 1 with an A residue at position 1 and an A residue at position 73. In still other aspects, the variant polypeptide comprises, consists essentially of, or consists of the amino acid sequence of SEQ ID NO: 1 with an A, D, and A residues at positions 1, 67 and 73. In still other aspects, the variant polypeptide comprises, consists essentially of, or consists of the amino acid sequence of SEQ ID NO: 1 with an A, H, and A residues at positions 1, 67 and 73. In still other aspects, the variant polypeptide comprises, consists essentially of, or consists of the amino acid sequence of SEQ ID NO: 1 with D and A residues positions 67 and 172. In still other aspects, the variant polypeptide comprises, consists essentially of, or consists of the amino acid sequence of SEQ ID NO: 1 with H and A residues at positions 67 and 172. n still other aspects, the variant polypeptide comprises, consists essentially of, or consists of the amino acid sequence of SEQ ID NO: 1 with A residues at positions 73 and 172. In still other aspects, the variant polypeptide comprises, consists essentially of, or consists of the amino acid sequence of SEQ ID NO: 1 with D, A and A residues at positions 67, 73 and 172. In still other aspects, the variant polypeptide comprises, consists essentially of, or consists of the amino acid sequence of SEQ ID NO: 1 with H, A and A residues at positions 67, 73 and 172. In still other aspects, the variant polypeptide comprises, consists essentially of, or consists of the amino acid sequence of SEQ ID NO: 1 with an A, D, A and A residues at positions 1, 67, 73 and 172. In still other aspects, the variant polypeptide comprises, consists essentially of, or consists of the amino acid sequence of SEQ ID NO: 1 with an A, H, A, and A residues at positions 1, 67, 73 and 172.

In some aspects, the variant polypeptide comprises, consists essentially of, or consists of the amino acid sequence of SEQ ID NO: 2 with an A residue at position 1. In other aspects, the variant polypeptide comprises, consists essentially of, or consists of the amino acid sequence of SEQ ID NO: 2 with an A residue at position 172. In yet other aspects, the variant polypeptide comprises, consists essentially of, or consists of the amino acid sequence of SEQ ID NO: 2 with an A residue at position 1 and 172. In other aspects, the variant polypeptide comprises, consists essentially of, or consists of the amino acid sequence of SEQ ID NO: 2 with a D residue at position 67. In yet other aspects, the variant polypeptide comprises, consists essentially of, or consists of the amino acid sequence of SEQ ID NO: 2 with a H residue at position 67. In still other aspects, the variant polypeptide comprises, consists essentially of, or consists of the amino acid sequence of SEQ ID NO: 2 with an A residue at position 73. In certain aspects, the variant polypeptide comprises, consists essentially of, or consists of the amino acid sequence of SEQ ID NO: 2 with a D residue at position 67 and an A residue at position 73. In yet other aspects, the variant polypeptide comprises, consists essentially of, or consists of the amino acid sequence of SEQ ID NO: 2 with a H residue at position 67 and an A residue at position 73. In still other aspects, the variant polypeptide comprises, consists essentially of, or consists of the amino acid sequence of SEQ ID NO: 2 with an A residue at position 1 and a D residue at position 67. In still other aspects, the variant polypeptide comprises, consists essentially of, or consists of the amino acid sequence of SEQ ID NO: 2 with an A residue at position 1 and a H residue at position 67. In still other aspects, the variant polypeptide comprises, consists essentially of, or consists of the amino acid sequence of SEQ ID NO: 2 with an A residue at position 1 and an A residue at position 73. In still other aspects, the variant polypeptide comprises, consists essentially of, or consists of the amino acid sequence of SEQ ID NO: 2 with an A, D, and A residues at positions 1, 67 and 73. In still other aspects, the variant polypeptide comprises, consists essentially of, or consists of the amino acid sequence of SEQ ID NO: 2 with an A, H, and A residues at positions 1, 67 and 73. In still other aspects, the variant polypeptide comprises, consists essentially of, or consists of the amino acid sequence of SEQ ID NO: 2 with D and A residues positions 67 and 172. In still other aspects, the variant polypeptide comprises, consists essentially of, or consists of the amino acid sequence of SEQ ID NO: 2 with H and A residues at positions 67 and 172. In still other aspects, the variant polypeptide comprises, consists essentially of, or consists of the amino acid sequence of SEQ ID NO: 2 with A residues at positions 73 and 172. In still other aspects, the variant polypeptide comprises, consists essentially of, or consists of the amino acid sequence of SEQ ID NO: 2 with D, A and A residues at positions 67, 73 and 172. In still other aspects, the variant polypeptide comprises, consists essentially of, or consists of the amino acid sequence of SEQ ID NO: 2 with H, A and A residues at positions 67, 73 and 172. In still other aspects, the variant polypeptide comprises, consists essentially of, or consists of the amino acid sequence of SEQ ID NO: 2 with an A, D, A and A residues at positions 1, 67, 73 and 172. In still other aspects, the variant polypeptide comprises, consists essentially of, or consists of the amino acid sequence of SEQ ID NO: 2 with an A, H, A, and A residues at positions 1, 67, 73 and 172.

The variant polypeptides having intein activity of the present disclosure, in some aspects, exhibit impaired splicing and/or impaired NTC at temperatures below 20° C. For example, the a variant polypeptide having intein activity may exhibit impaired spicing and/or impaired NTC at a temperature of less than about 20° C., less than about 19° C., less than about 18° C., less than about 17° C., less than about 16° C., less than about 15° C., less than about 14° C., less than about 13° C., less than about 12° C., less than about 11° C., or less than about 10° C. In some cases, the variant polypeptide having intein activity exhibits impaired splicing and/or impaired NTC at a temperature of about 5° C., about 6° C., about 7° C., about 8° C., about 9° C., about 10° C., about 11° C., about 12° C., about 13° C., about 14° C., about 15° C., about 16° C., about 17° C., about 18° C., or about 19° C.

The variant polypeptides having intein activity of the present disclosure, in other aspects, exhibit controllable splicing and/or N-terminal cleavage at temperatures ranging from about 20° C. to about 50° C. For example, the a variant polypeptide having intein activity may exhibit controllable spicing and/or NTC at a temperature ranging from about 20° C. to about 50° C., from about 21° C. to about 49° C., from about 22° C. to about 48° C., from about 23° C. to about 47° C., from about 24° C. to about 46° C., from about 25° C. to about 45° C., from about 26° C. to about 44° C., from about 27° C. to about 43° C., from about 28° C. to about 42° C., from about 29° C. to about 41° C., from about 30° C. to about 40° C., or from about 31° C. to about 39° C. In some cases, the variant polypeptide having intein activity exhibits controllable splicing and/or NTC at a temperature of about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

### Fusion Proteins

Aspects of the present disclosure relate to fusion proteins comprising the polypeptides having intein activity of the present disclosure.

In an aspect, a fusion protein comprising from N- to C-terminus an N-terminal extein polypeptide, an intein polypeptide, and a C-terminal extein polypeptide, wherein the intein polypeptide comprises a polypeptide having intein activity of the present disclosure.

The fusion proteins of the present disclosure contemplate the use of an N-terminal extein polypeptide and any C-terminal extein polypeptide.

In some aspects, the N-terminal extein polypeptide comprises a protein of interest (e.g., a protein selected for expression and/or purification).

The N-terminal extein polypeptide can include an N-terminal and/or C-terminal affinity tag, for example, to facilitate purification of the protein of interest. In some aspects, the N-terminal extein polypeptide comprises an N-terminal affinity tag. The disclosure contemplates the use of any affinity tag available to the skilled artisan. In some embodiments, the N-terminal affinity tag comprises a His-tag (e.g., polyhistidine).

The intein variant polypeptides of the present disclosure can accommodate a variety of N-terminal extein polypeptides having any residue at the −1 position. Surprisingly, the residue at the −1 position of the N-terminal extein polypeptide can selected to control the rate of NTC. In some aspects, the −1 position of the N-terminal extein polypeptide is selected from a D, H, or K residue for fast NTC wherein at least 50% of the NTC occurs at 37° C. within about 2 hours. In other aspects, the −1 position of the N-terminal extein polypeptide is selected from a E, F, G, L, M, Q, R, W or Y amino acid residue for moderate NTC wherein at least 50% of the NTC occurs at 37° C. within about 6 hours. In yet other aspects, the −1 position of the N-terminal extein polypeptide is selected from an A, C, I, N, P, S, T, or V amino acid residue for slow NTC wherein less than about 50% of the NTC occurs at 37° C. after about 6 hours.

In an embodiment, the residue at the −1 position of the N-terminal extein polypeptide is a D residue. In another embodiment, the residue at the −1 position of the N-terminal extein polypeptide is a H residue. In another embodiment, the residue at the −1 position of the N-terminal extein polypeptide is a K residue. In another embodiment, the residue at the −1 position of the N-terminal extein polypeptide is an E residue. In another embodiment, the residue at the −1 position of the N-terminal extein polypeptide is a F residue. In another embodiment, the residue at the −1 position of the N-terminal extein polypeptide is a G residue. In another embodiment, the residue at the −1 position of the N-terminal extein polypeptide is a L residue. In another embodiment, the residue at the −1 position of the N-terminal extein polypeptide is a M residue. In another embodiment, the residue at the −1 position of the N-terminal extein polypeptide is a Q residue. In another embodiment, the residue at the −1 position of the N-terminal extein polypeptide is a R residue. In another embodiment, the residue at the −1 position of the N-terminal extein polypeptide is a W residue. In another embodiment, the residue at the −1 position of the N-terminal extein polypeptide is a Y residue. In another embodiment, the residue at the −1 position of the N-terminal extein polypeptide is a A residue. In another embodiment, the residue at the −1 position of the N-terminal extein polypeptide is a C residue. In another embodiment, the residue at the −1 position of the N-terminal extein polypeptide is an I residue. In another embodiment, the residue at the −1 position of the N-terminal extein polypeptide is a N residue. In another embodiment, the residue at the −1 position of the N-terminal extein polypeptide is a P residue. In another embodiment, the residue at the −1 position of the N-terminal extein polypeptide is a S residue. In another embodiment, the residue at the −1 position of the N-terminal extein polypeptide is a T residue. In another embodiment, the residue at the −1 position of the N-terminal extein polypeptide is a V residue.

In some aspects, the C-terminal extein polypeptide comprises a reporter polypeptide.

### Systems

Aspects of the present disclosure relate to intein-based expression and purification systems comprising the polypeptides and/or fusion proteins of the present disclosure.

In an aspect, an intein-based protein expression and purification system, comprises: (a) a polypeptide having intein activity of the present disclosure or a fusion protein of the present disclosure; (b) a host cell comprising an expression vector or construct encoding the polypeptide or the fusion protein for expression of a protein of interest; and (c) a chromatography resin for purification of the protein of interest.

The disclosure contemplates the use of any suitable host cell. Exemplary host cells include, archaeal host cells, bacterial host cells, fungal host cells, and yeast host cells.

Exemplary archaeal host cells include, without limitation, Methanococcus maripaludis, Halobacterium salinarum, Pyrococcus furiosus, and Methanothermobacter thermautotrophicus.

Exemplary bacterial host cells include, without limitation, Escherichia coli, Bacillus subtilis, Pseudomonas fluorescens, Corynebacterium glutamicum.

Exemplary fungal host cells include, without limitation, Aspergillus niger, Trichoderma reesei, and Neurospora crassa.

Exemplary yeast host cells include, without limitation, Candida glabrata, Hanseniaspora uvarum, Kluyveromyces lactis, Pichia pastoris, and Saccharomyces cerevisiae.

Aspects of the present disclosure relate to polynucleotides encoding a polypeptide having intein activity of the present disclosure, fusion proteins comprising the polypeptides, and expression vectors or nucleic acid constructs comprising the polynucleotides.

In some aspects, the expression vector or construct is operably linked to an inducible promoter.

In an exemplary aspect, the system comprises an E. coli host cell and the inducible promoter is the T7 promoter.

In some aspects, the system comprises an inducer for promoting expression of the protein of interest. An exemplary inducer is isopropyl b-D-1-thiogalactopyrandoside.

In some aspects, the chromatography resin comprises metal affinity chromatography beads and the N-terminal affinity tag comprises a His-tag.

In some aspects, the system comprises a nucleophile to increase the rate of NTC. In some aspects, the nucleophile is DTT. In other aspects, the nucleophile is not DTT. In still other aspects, the nucleophile is HA. In some aspects, the system does not comprise an external nucleophile.

### Kits

Aspects of the present disclosure relate to kits comprising the polypeptides, the fusion proteins, and/or systems of the present disclosure, for example, for use in implementing a process of the present disclosure.

In an aspect, the present disclosure provides a kit comprising a polypeptide having intein activity of the present disclosure, a fusion protein of the present disclosure, a system of the present disclosure, and instructions for using the polypeptide, the fusion protein, or the system for intein-based purification of a protein of interest.

### Processes

Aspects of the present disclosure relate to processes for separating a protein of interest on a chromatography resin. In an aspect, a process for separating of a protein of interest on a chromatography resin comprises: (a) contacting a fusion protein of the present disclosure optionally expressed using a system of the present disclosure with a chromatography resin to produce a chromatography resin-bound fusion protein, wherein contacting is performed at temperatures below 20° C. reduce or prevent NTC of the N-terminal extein polypeptide while the fusion protein remains bound to the chromatography resin; (b) incubating the chromatography resin-bound fusion protein at temperature greater than or equal to about 20° C. to initiate NTC of the N-terminal extein polypeptide and release the polypeptide having intein activity and C-terminal extein polypeptide so that the N-terminal extein polypeptide remains bound to the chromatography resin; and (c) recovering the N-terminal extein polypeptide from the chromatography resin.

### EXAMPLES

The present disclosure has multiple aspects, illustrated by the non-limiting examples as described herein.

### Materials & Methods

### Plasmids

All reporter constructs described in this manuscript were mutagenized, sequenced, and prepared commercially (Genscript) using the previously described MIG reporter backbone (Weinberger II and Lennon 2021), which is resistant to chloramphenicol and under the control of a T7-inducible promoter.

### Protein Expression

Following plasmid transformation into Escherichia coli BL21 (DE3) (New England Biolabs), cells were grown with shaking at 250 rpm in LB broth with 25 mg/mL chloramphenicol at 37° C. to an optical density of approximately 0.5 at 600 nm, culture temperature was reduced to 15° C. with shaking at 250 rpm continued, and protein expression was induced by addition of 1 mM isopropyl b-D-1-thiogalactopyrandoside. After approximately 20 hours of protein expression at 15° C., cultures were centrifuged at 4000×g to harvest cells.

### Splicing and N-Terminal Cleavage Assays

Following protein expression, cell pellets were suspended in 1×PBS (11.9 mM phosphates, 137 mM sodium chloride, 2.7 mM potassium chloride, pH 7.4), lysed by sonication, and insoluble material was removed by centrifugation at 16,100×g. Whole cell supernatant (WCS) containing the expressed MIG protein was used to monitor splicing or NTC. For time zero (TO), WCS was mixed with Bio-Rad 4× Laemmli sample buffer following centrifugation and stored at −20° C. To purify the MIG protein referred to as TkPl-AA, a construct where the HEN adjacent residues of Tk RadA intein were mutated to match those of the HEN-lacking Pyrococcus horikoshii RadA mini-intein (described further in Results) for NTC analyses, cell pellets were resuspended in 20 mM Tris (pH 8.0), 30 mM imidazole, and 500 mM sodium chloride. Following lysis by sonication, MIG TkPl-AA was isolated via an N-terminal His-tag from WCS using Ni-Charged MagBeads (Genscript). Purified protein was eluted in 20 mM Tris (pH 8.0), 500 mM imidazole, and 500 mM sodium chloride, then dialyzed into 1×PBS prior to NTC assays. For all splicing and NTC assays, including on-column cleavage, samples were incubated at the temperature and time indicated in Results and Figures. To stop reactions, samples were mixed with Bio-Rad 4× Laemmli sample buffer and stored at −20° C. Where applicable, ZnCl2 was present at 10 mM and external nucleophiles (DTT and HA) were present at 50 mM.

### SDS-PAGE

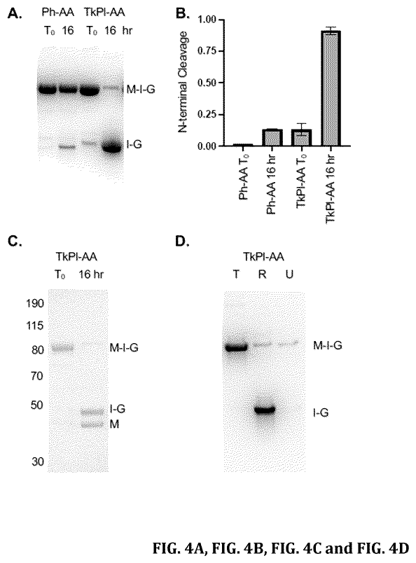

In all assays except FIG. 4C, in-gel fluorescence is used to visualize GFP-containing fluorescent products following semi-native PAGE as described (Weinberger II and Lennon 2021). Briefly, reaction mixtures were separated using Tris-glycine TGX gels (Bio-Rad) where samples were not heated prior to separation in order to maintain GFP structure. Coomassie staining was used to visualize all products in FIG. 4C.

### Imaging and Data Analysis

GFP-containing products were observed using in-gel fluorescence measurements immediately following SDS-PAGE by an Amersham Imager 680 (GE Healthcare). Coomassie-stained products were visualized using white light. In all cases, ImageJ (available online) was used to measure relative band amounts by densitometry. Average NTC or splicing, as well as standard deviation, was calculated at each time point by averaging three independent reactions and plotted using Prism (GraphPad).

### Example 1—Conditional Protein Splicing is Possible at Mesophilic Temperatures

A variant of an intein naturally located within the T. kodakarensis RadA protein that displayed unique activity when placed within a splicing reporter construct and expressed within E. coli was identified and characterized. Deletion of the homing endonuclease domain (residues 276-585) from the native intein generated a variant (referred to as TkDE) that was defective for splicing within our reporter construct when expressed at low temperature (15° C.), but that spliced efficiently at modest temperatures (50° C.) (Liman et al. 2024). Given the potential to exploit TkDE for protein purification, the temperature-dependent rescue of TkDE splicing was probed in more detail, reasoning that, if splicing could be activated at modest temperatures, then TkDE could be useful for intein applications at temperatures that are physiologically relevant for many model organisms.

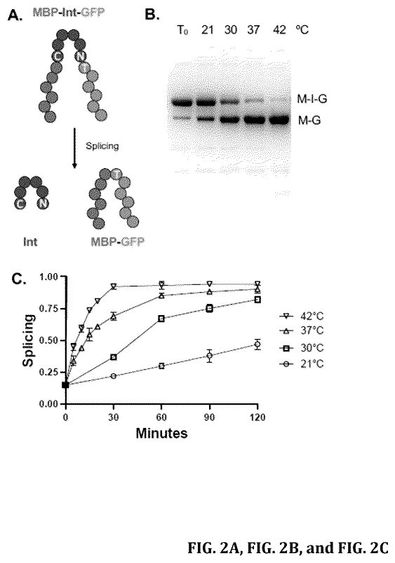

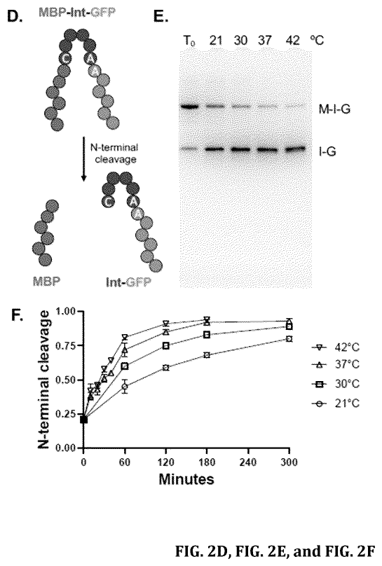

Splicing and cleavage efficiencies were measured using a reporter referred to as MIG (MBP-Intein-GFP). In this reporter, TkDE (with 10 N-terminal and 10 C-terminal residues from the native RadA exteins) is flanked by the E. coli maltose binding protein (MBP) with an N-terminal His-tag (His-MBP) as the N-extein and the superfolder green fluorescent protein (GFP) as the C-extein (FIG. 2A) (Topilina et al. 2015). Precursor and splicing or cleavage product ratios can be monitored by in-gel fluorescence following semi-native PAGE (Weinberger II and Lennon 2021). Note that MIG assays are performed within crude cell supernatants unless otherwise described. Following expression for 20 hours in E. coli at 15° C. (TO), it was observed that very little splicing had occurred in vivo, with approximately 85% fluorescent products as unspliced precursor (FIG. 2B, FIG. 2C). In contrast to poor splicing efficiencies observed in vivo at 15° C. during protein expression and cell lysis, incubation of the TkDE intein in the MIG reporter at 37° C. and 42° C. resulted in increased and then near total splicing within one hour. At 30° C., approximately half of the precursor had spliced after one hour (FIG. 2B and FIG. 2C). Splicing was possible but substantially slower at 21° C., with approximately 25% of the precursor converting to ligated exteins after two hours (FIG. 2B and FIG. 2C).

### Example 2—TkDE Permits Conditional NTC at Mesophilic Temperatures

NTC is particularly useful for intein-mediated affinity tag removal from a protein of interest following purification. However, conditional splicing by an intein does not necessarily mean that NTC will proceed under similar conditions, particularly without an external nucleophile. Therefore, it was examined whether TkDE could undergo NTC in response to temperature in a manner similar to splicing. To promote NTC instead of splicing, the last residue of the intein, a conserved Asn, and the first residue of the C-extein, a conserved Thr, were changed to Ala (TkDE-AA; FIG. 2D). After expression at 15° C. in E. coli for 20 hours, only approximately 25% of the precursor had undergone NTC to form His-MBP and intein-GFP (FIG. 2E). Incubation of TkDE-AA in crude lysates lacking an external nucleophile at temperatures ranging from 21° C. to 42° C. stimulated NTC. As with TkDE splicing, higher temperatures led to increasing rates of NTC for TkDE-AA (FIG. 2E and FIG. 2F).

### Example 3—TkDE Protein Splicing and TkDE-AA NTC is Inhibited by Zinc

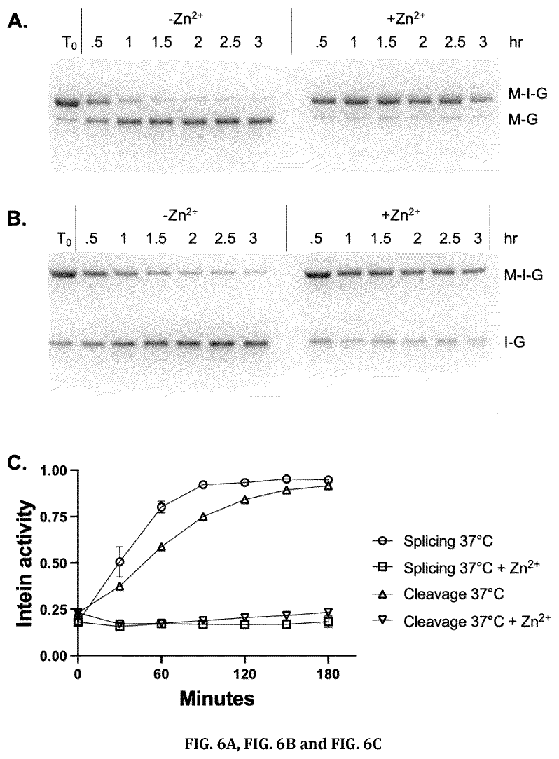

Zinc is known to be a potent inhibitor of intein-mediated protein splicing; however, this inhibition can be reversed by introducing a chelator, such as ethylenediaminetetraacetic acid (EDTA) (Mills et al. 2001; Woods et al. 2020). Inhibition can be mediated through direct binding of zinc to the initiating nucleophilic residue of the intein required for splicing and NTC (Woods et al. 2020). It was then examined whether zinc addition could diminish or inhibit the splicing and NTC activities of of TkDE and TkDE-AA, respectively. As expected, the addition of just 10 mM zinc is sufficient to radically diminish both splicing and NTC (FIG. 6A, FIG. 6B, and FIG. 6C), providing a second mechanism (with temperature control as the first) to control conditional protein splicing and NTC activities of the TkDE intein.

### Example 4—the TkDE Intein can Efficiently Drive NTC without an External Nucleophile

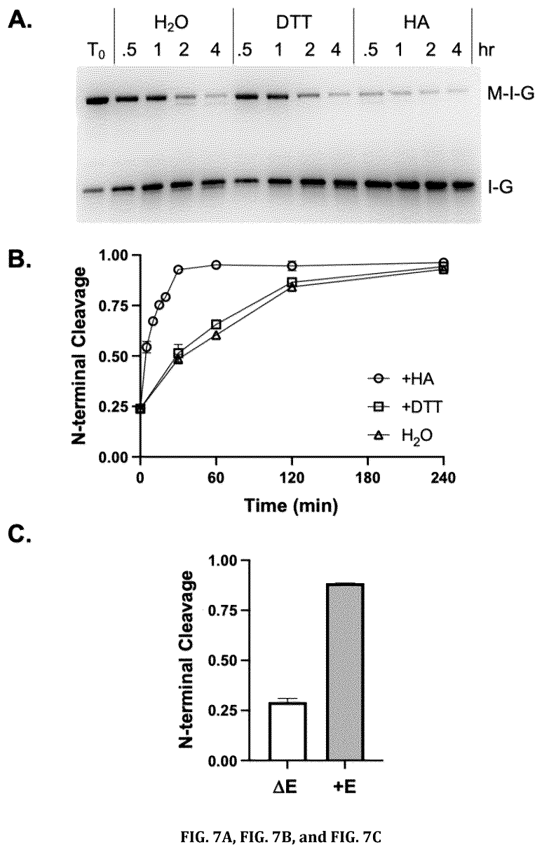

For existing intein-based protein purification schemes utilizing NTC as a means of affinity tag removal, efficient NTC can be driven at lower temperatures by the addition of high concentrations of an external nucleophile. One commonly used nucleophile is DTT, which promotes thiolysis of the scissle thioester bond between the N-extein and intein (Prabhala et al. 2022). However, DTT reduces disulfide bonds that may be required for proper folding of some proteins and the required concentrations would be prohibitively expensive for large-scale preparations, limiting the value of driving NTC through DTT addition. Therefore, an intein capable of NTC in the absence of DTT could be useful for certain applications. Excitingly, TkDE-AA can efficiently undergo NTC in the absence of an external nucleophile (e.g. DTT) (FIG. 7A and FIG. 7B) at mesophilic temperatures. The rates of NTC were compared in the presence and absence of DTT and, surprisingly, found that the rate of cleavage is not accelerated by DTT (FIG. 7A and FIG. 7B). Interestingly, the smaller external nucleophile hydroxylamine (HA), which is also known to accelerate the rate of NTC (Amitai et al. 2009), increases the cleavage rate by approximately 5-fold (FIG. 7A and FIG. 7B). Therefore, the smaller external nucleophile HA, but not the larger DTT, is capable of accelerating NTC.

### Example 5—HEN Domain Deletion does not Inhibit NTC

Given the observation that DTT does not stimulate TkDE-AA NTC, there was an interest in determining whether this was an inherent property of the T. kodakarensis RadA intein or due to deletion of the HEN domain. It was previously found that splicing was robust for the T. kodakarensis RadA intein with the HEN domain present, with greater than 90% splicing during expression in E. coli at 15° C. (Liman et al. 2024). Note that the HEN domain's active site (residues 373-381) was mutated to prevent DNA cleavage and toxicity to E. coli. To determine whether TkE could perform NTC, the terminal Asn of the intein and +1 residue of the C-extein were mutated (which are referred to here as TkE-AA), which is capable of NTC but not splicing. This variant is identical to TkDE-AA, except for the presence of the HEN domain. Following overnight expression in E. coli at 15° C., it was found that that approximately 90% of TkDE-AA has undergone NTC, compared to just 25% for TkDE-AA (FIG. 7C). These results are consistent with previous observations that the T. kodakarensis RadA intein activity increases when the HEN domain is present (Liman et al. 2024).

### Example 6—Each Amino Acid at the −1 Position Supports Controlled and Efficient NTC

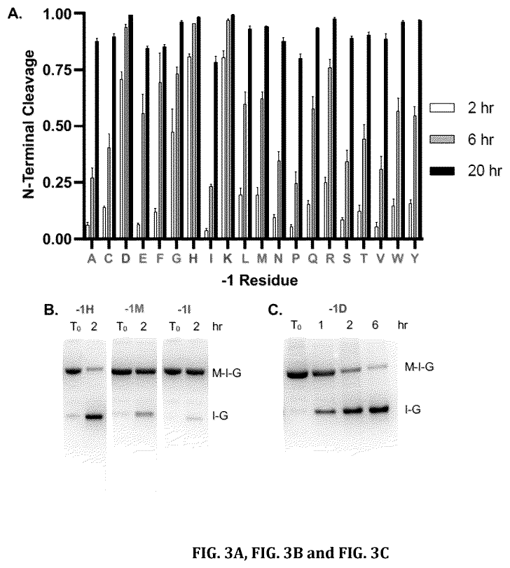

The identity of the last residue of the N-extein, known as the −1 position, has been shown to drastically influence the rate of NTC (Amitai et al. 2009). We, therefore, investigated how the identity of the −1-residue affected NTC by TkDE-AA in the MIG context, examining all twenty amino acids in the −1 position after a 2, 6, or 20-hour incubation at 37° C. As expected, the rate of NTC was highly variable depending on the identity of the residue in the −1 position, with the native-1 residue for TkDE-AA, lysine, displaying the fastest NTC rate (FIG. 3A). The twenty-1 TkDE-AA residue variants were classified into fast, moderate, or slow based on NTC rate, with fast demonstrating greater than 50% NTC after 2 hours, moderate greater than 50% NTC after 6 hours, and slow less than 50% NTC after 6 hours (FIG. 3A). In addition to lysine, only histidine and aspartate fell into the fast category, with nine residues displaying a relatively moderate rate of NTC (glutamate, phenylalanine, glycine, leucine, methionine, glutamine, arginine, tryptophan, tyrosine), and the remaining eight residues undergo NTC at a relatively slow rate (alanine, cysteine, isoleucine, asparagine, proline, serine, threonine, valine) (FIG. 3A). An example from each rate category following incubation at 37° C. for two hours is shown (FIG. 3B). A clear pattern of NTC rate based on the physical nature of the −1-position side chain was not readily apparent. Importantly, while the NTC rate is highly variable depending on the −1 position, all residues display nearly complete NTC following a 20-hour incubation at 37° C.

### Example 7—TkDE-AA Permits Controlled NTC with Aspartate at the −1 Position

For many inteins, an aspartate at the −1 position leads to premature NTC, even if the construct is capable of productive splicing (Oeemig et al. 2012; Amitai et al. 2009). For example, the Pyrococcus horikoshii (Ph) RadA intein primarily undergoes NTC, rather than splicing, with aspartate as the −1 residue, even without mutations to favor NTC (Oeemig et al. 2012). This effectively excludes the use of intein-based purification strategies to proteins that do not end in aspartate. Interestingly, it was demonstrated that for TkDE-AA, aspartate in the −1 position does not lead to premature cleavage (FIG. 3C). Following expression in E. coli for 20 hours at 15° C., less than 5% NTC has occurred (FIG. 3C). After incubation at 37° C., NTC proceeds rapidly, with nearly 75% NTC after only 2 hours (FIG. 3C).

### Example 8—TkDE-AA can be Minimized without Compromising Functionality

For TkDE-AA to be more useful as a potential means for affinity tag removal, the number of residues dividing the N-extein from the intein should be minimized. The MIG reporter has a spacer between MBP and the intein that includes a Factor Xa cleavage site and ten residues from the native N-extein. To investigate whether these sequences could be removed without compromising NTC, these residues were eliminated, fusing the His-MBP tag with a single lysine residue on the C-terminus to TkDE-AA. To compensate for any potential loss in intein activity, the residues surrounding the homing endonuclease insertion site of the Tk RadA intein were changed to match those of the Ph RadA intein, a mini-intein that naturally lacks a homing endonuclease. It was previously found that these substitutions increase TkDE splicing (Liman et al. 2024). This intein is referred to as TkPl-AA. A comparison of the TkDE and Ph RadA intein residues is provided (FIG. 8).

Following TkPl-AA expression at 15° C. for 20 hours, minimal NTC is observed (FIG. 4A and FIG. 4 B), yet, upon incubation of TkPl-AA in the absence of an external nucleophile at 37° C. for 16 hours, greater than 90% NTC was found to have occurred (FIG. 4A and FIG. 4B). The question was asked whether the ability to perform NTC under these conditions was unusual compared to a similar intein. To answer this question, the NTC rates of TkPl-AA were compared to the RadA intein from P. horikoshii. Both the Ph and TkPl RadA inteins are the same length and are greater than 75% identical in residue sequence. When incubating the Ph RadA intein, with the terminal asparagine of the intein and +1 threonine mutated to alanine, and in an identical extein context (Ph-AA), minimal NTC was observed compared to TkPl-AA (FIG. 4A and FIG. 4B). Interestingly, while Ph-AA NTC is inhibited compared to TkPl-AA, it was previously found that splicing of the Ph RadA intein is more efficient than the TkPl intein (Liman et al. 2024).

### Example 9—N-Terminal Cleavage of Purified TkPl-AA

TkPl-AA was evaluated in a real-world context as a potential tool for affinity tag removal, a common application for inteins, but one that currently requires high concentrations of DTT or extended incubation above 50° C. Following purification using immobilized metal affinity chromatography, the TkPl-AA MIG precursor (His-MBP-intein-GFP) can be isolated with minimal NTC (FIG. 4C), which is ideal as premature cleavage reduces purification yields. While the elimination of the spacer between MBP and the intein slows the rate of NTC for TkPl-AA compared to Tk Δ E-AA, this largely blocks premature NTC prior to and during purification (T0; FIG. 4C). Following incubation at 37° C. for 16 hours at neutral pH in the absence of an external nucleophile, virtually all precursor underwent NTC, resulting in two bands corresponding in size to His-MBP (42.6 kDa) and TkPl-AA-GFP (47.8 kDa). These bands are both visible following Coomassie staining (FIG. 4C).

During purification, the TkPl-AA MIG precursor is bound to metal affinity chromatography beads via an N-terminal His-tag (FIG. 4D)). From this point, the precursor can either be eluted, or can remain bound to the beads and incubated at 37° C. to induce NTC. When TkPl-AA precursor is bound to the beads and incubated at 37° C. for 24 hours, a majority of the precursor remains active, undergoing NTC (FIG. 4D). This results in release of intein-GFP into solution during the incubation, whereas His-MBP remains bound to the beads. This provides a useful strategy for separating a target protein (e.g., His-MBP) from unwanted components (e.g., intein-GFP) directly on affinity beads (FIG. 4D)

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

For reasons of completeness, various aspects of the disclosure are set out in the following numbered clauses:

Clause 1. A polypeptide having intein activity, comprising an amino acid sequence having at least about 80%, at least about 85%, at least about 90%, at least about 91%, at least about 92%, at least about 93%, at least about 94%, at least about 95%, at least about 96%, at least about 97%, at least about 98%, or at least about 99%, but less than 100%, sequence identity to the amino acid sequence of SEQ ID NO: 1 (TKΔE) or SEQ ID NO: 2 (TkPl), wherein the polypeptide has intein activity.

Clause 2. The polypeptide of clause 1, further comprising an A residue at a position corresponding to position 1 and/or a position corresponding to position 172 of SEQ ID NO: 1 or SEQ ID NO: 2.

Clause 3. The polypeptide of clause 1 or clause 2, further comprising an A residue at position 1 and/or at position 172 of SEQ ID NO: 1 or SEQ ID NO: 2.

Clause 4. The polypeptide of any one of clauses 1-3, further comprising a D residue at a position corresponding to position 67 and/or an A residue at a position corresponding to position 73 of SEQ ID NO: 1 or SEQ ID NO: 2.

Clause 5. The polypeptide of any one of clauses 1-4, further comprising a D residue at a position 67 and/or an A residue at position 73 of SEQ ID NO: 1 or SEQ ID NO: 2.

Clause 6. The polypeptide of any one of clauses 1-5, further comprising a H residue at a position corresponding to position 67 and/or an A residue at a position corresponding to position 73 of SEQ ID NO: 1 or SEQ ID NO: 2.

Clause 7. The polypeptide of any one of clauses 1-6, further comprising a H residue at a position 67 and/or an A residue at position 73 of SEQ ID NO: 1 or SEQ ID NO: 2.

Clause 8. The polypeptide of any one of clauses 1-7, further comprising an A residue, a D residue, an A residue and/or an A residue at positions corresponding to positions 1, 67, 73 and/or 172 of SEQ ID NO: 1 or SEQ ID NO: 2.

Clause 9. The polypeptide of any one of clauses 1-8, further comprising an A residue, a H residue, an A residue and/or an A residue at positions corresponding to positions 1, 67, 73 and/or 172 of SEQ ID NO: 1 or SEQ ID NO: 2.

Clause 10. The polypeptide of any one of clauses 1-9, further comprising an A residue, a D residue, an A residue and/or an A residue at positions 1, 67, 73 and/or 172 of SEQ ID NO: 1 or SEQ ID NO: 2.

Clause 11. The polypeptide of any one of clauses 1-10, further comprising an A residue, a H residue, an A residue and/or an A residue at positions 1, 67, 73 and/or 172 of SEQ ID NO: 1 or SEQ ID NO: 2.

Clause 12. The polypeptide of any one of clauses 1-11, wherein the polypeptide has at least 90%, but less than 100%, sequence identity to the amino acid sequence of SEQ ID NO: 1 or SEQ ID NO: 2.

Clause 13. The polypeptide of any one of clauses 1-12, wherein the polypeptide has at least 91%, but less than 100%, sequence identity to the amino acid sequence of SEQ ID NO: 1 or SEQ ID NO: 2.

Clause 14. The polypeptide of any one of clauses 1-13, wherein the polypeptide has at least 92%, but less than 100%, sequence identity to the amino acid sequence of SEQ ID NO: 1 or SEQ ID NO: 2.

Clause 15. The polypeptide of any one of clauses 1-14, wherein the polypeptide has at least 93%, but less than 100%, sequence identity to the amino acid sequence of SEQ ID NO: 1 or SEQ ID NO: 2.

Clause 16. The polypeptide of any one of clauses 1-15, wherein the polypeptide has at least 94%, but less than 100%, sequence identity to the amino acid sequence of SEQ ID NO: 1 or SEQ ID NO: 2.

Clause 17. The polypeptide of any one of clauses 1-16, wherein the polypeptide has at least 95%, but less than 100%, sequence identity to the amino acid sequence of SEQ ID NO: 1 or SEQ ID NO: 2.

Clause 18. The polypeptide of any one of clauses 1-17, wherein the polypeptide has at least 96%, but less than 100%, sequence identity to the amino acid sequence of SEQ ID NO: 1 or SEQ ID NO: 2.

Clause 19. The polypeptide of any one of clauses 1-18, wherein the polypeptide has at least 97%, but less than 100%, sequence identity to the amino acid sequence of SEQ ID NO: 1 or SEQ ID NO: 2.

Clause 20. The polypeptide of any one of clauses 1-19, wherein the polypeptide has at least 98%, but less than 100%, sequence identity to the amino acid sequence of SEQ ID NO: 1 or SEQ ID NO: 2.

Clause 21. The polypeptide of any one of clauses 1-20, wherein the polypeptide has at least 99%, but less than 100%, sequence identity to the amino acid sequence of SEQ ID NO: 1 or SEQ ID NO: 2.

Clause 22. The polypeptide of any one of clauses 1-21, wherein the polypeptide comprises, consists essentially of, or consists of:
(a) the amino acid sequence of SEQ ID NO: 1 with an A residue at positions 1, a D or an H residue at position 67, an A residue at position 73, and/or an A residue at position 172;
(b) the amino acid sequence of SEQ ID NO: 2 with an A residue at positions 1, a D or an H residue at position 67, an A residue at position 73, and/or an A residue at position 172.

Clause 23. The polypeptide of any one of clauses 1-22, wherein:
(a) the polypeptide exhibits impaired splicing and N-terminal cleavage (NTC) at temperatures below 20° C.; and/or
(b) the polypeptide exhibits controllable splicing and N-terminal cleavage at temperatures ranging from about 20° C. to about 50° C.

Clause 24. A fusion protein comprising from N- to C-terminus an N-terminal extein polypeptide, an intein polypeptide, and a C-terminal extein polypeptide, wherein the intein polypeptide comprises the polypeptide having intein activity of any one of clauses 1-23.

Clause 25. The fusion protein of clause 24, wherein the residue at the −1 position of the N-terminal extein polypeptide is selected to control the rate of NTC, wherein:
(a) the −1 position of the N-terminal extein polypeptide is selected from a D, H, or K residue for fast NTC wherein at least 50% of the NTC occurs at 37° C. within about 2 hours;
(b) the −1 position of the N-terminal extein polypeptide is selected from a E, F, G, L, M, Q, R, W or Y residue for moderate NTC wherein at least 50% of the NTC occurs at 37° C. within about 6 hours; or
(c) the −1 position of the N-terminal extein polypeptide is selected from a A, C, I, N, P, S, T, or V residue for slow NTC wherein less than about 50% of the NTC occurs at 37° C. after about 6 hours.

Clause 26. The fusion protein of clause 24 or clause 25, wherein the residue at the −1 position of the N-terminal extein polypeptide is a D residue.

Clause 27. The fusion protein of any one of clauses 24-26, wherein the N-terminal extein polypeptide comprises a protein of interest.

Clause 28. The fusion protein of any one of clauses 24-27, wherein the N-terminal extein polypeptide comprises an N-terminal affinity tag.

Clause 29. The fusion protein of any one of clauses 24-28, wherein the C-terminal extein polypeptide comprises a reporter polypeptide.

Clause 30. An intein-based protein expression and purification system, comprising:
(a) the polypeptide having intein activity of any one of clause 1-22 or the fusion protein of any one of clauses 24-29;
(b) a host cell comprising an expression vector or construct encoding the polypeptide or the fusion protein for expression of a protein of interest; and
(c) a chromatography resin for purification of the protein of interest.

Clause 31. The system of clause 30, wherein the expression vector or construct is operably linked to an inducible promoter.

Clause 32 The system of clause 31, further comprising an inducer for promoting expression of the protein of interest.

Clause 33. The system of any one of clauses 30-32, wherein the chromatography resin comprises metal affinity chromatography beads and the N-terminal affinity tag comprises a His-tag.

Clause 34. The system of any one of clauses 30-33, further comprising a nucleophile to increase the rate of NTC.

Clause 35. A kit comprising the polypeptide having intein activity of any one of clauses 1-22, the fusion protein of any one of clauses 24-29, or the system of any one of clauses 30-34, and instructions for using the polypeptide, the fusion protein, or the system for intein-based purification of a protein of interest.

Clause 36. A process for separating of a protein of interest on a chromatography resin, comprising:
(a) contacting the fusion protein of any one of clauses 24-29 optionally expressed using the system of any one of clauses 30-34 with a chromatography resin to produce a chromatography resin-bound fusion protein, wherein contacting is performed at temperatures below 20° C. reduce or prevent NTC of the N-terminal extein polypeptide while the fusion protein remains bound to the chromatography resin;
(b) incubating the chromatography resin-bound fusion protein at temperature greater than or equal to 20° C. to initiate NTC of the N-terminal extein polypeptide and release the polypeptide having intein activity and C-terminal extein polypeptide so that the N-terminal extein polypeptide remains bound to the chromatography resin; and
(c) recovering the N-terminal extein polypeptide from the chromatography resin.

### REFERENCES

Amitai, G., Callahan, B. P., Stanger, M. J., Belfort, G., and Belfort, M. (2009). Modulation of intein activity by its neighboring extein substrates. Proc Natl Acad Sci USA 106, 11005-11010. doi: 10.1073/pnas.0904366106.
Buskirk, A. R., Ong, Y.-C., Gartner, Z. J., and Liu, D. R. (2004). Directed evolution of ligand dependence: small-molecule-activated protein splicing. Proc Natl Acad Sci USA 101, 10505-10510. doi: 10.1073/pnas.0402762101.
Lennon, C. W., and Belfort, M. (2017). Inteins. Curr Biol 27, R204-R206. doi: 10.1016/j.cub.2017.01.016.
Liman, G. L. S., Lennon, C. W., Mandley, J. L., Galyon, A. M., Zatopek, K. M., Gardner, A. F., Santangelo, T. J. (2024). Intein-splicing can control archaeal DNA replication. Sci Adv 10, eadp4995. doi: 10.1126/sciadv.adp4995
Mills, K. V., Connor, K. R., Dorval, D. M., and Lewandowski, K. T. (2006). Protein purification via temperature-dependent, intein-mediated cleavage from an immobilized metal affinity resin. Anal Biochem 356, 86-93. doi: 10.1016/j.ab.2006.04.055.
Mills, K. V., Johnson, M. A., and Perler, F. B. (2014). Protein splicing: how inteins escape from precursor proteins. J Biol Chem 289, 14498-14505. doi: 10.1074/jbc.R113.540310.
Mills, K. V., Manning, J. S., Garcia, A. M., and Wuerdeman, L. A. (2004). Protein splicing of a Pyrococcus abyssi intein with a C-terminal glutamine. J Biol Chem 279, 20685-20691. doi: 10.1074/jbc.M400887200.
Mills, K. V., and Paulus, H. (2001). Reversible Inhibition of Protein Splicing by Zinc Ion. Journal of Biological Chemistry 276, 10832-10838. doi: 10.1074/jbc.M011149200.
Oeemig, J. S., Zhou, D., Kajander, T., Wlodawer, A., and Iwaï, H. (2012). NMR and crystal structures of the Pyrococcus horikoshii RadA intein guide a strategy for engineering a highly efficient and promiscuous intein. J Mol Biol 421, 85-99. doi: 10.1016/j.jmb.2012.04.029.
Prabhala, S. V., Gierach, I., and Wood, D. W. (2022). The Evolution of Intein-Based Affinity Methods as Reflected in 30 years of Patent History. Front Mol Biosci 9, 857566. doi: 10.3389/fmolb.2022.857566.
Sarmiento, C., and Camarero, J. A. (2019). Biotechnological Applications of Protein Splicing. Curr Protein Pept Sci 20, 408-424. doi: 10.2174/1389203720666190208110416.
Skretas, G., and Wood, D. W. (2005). Regulation of protein activity with small-molecule-controlled inteins. Protein Sci 14, 523-532. doi: 10.1110/ps.04996905.
Topilina, N. I., Green, C. M., Jayachandran, P., Kelley, D. S., Stanger, M. J., Piazza, C. L., et al. (2015). SufB intein of Mycobacterium tuberculosis as a sensor for oxidative and nitrosative stresses. Proc Natl Acad Sci USA 112, 10348-10353. doi: 10.1073/pnas. 1512777112.
Weinberger II, J., and Lennon, C. W. (2021). Monitoring Protein Splicing Using In-gel Fluorescence Immediately Following SDS-PAGE. Bio Protoc 11, e4121. doi: 10.21769/BioProtoc.4121.
Wood, D. W., Belfort, M., and Lennon, C. W. (2023). Inteins-mechanism of protein splicing, emerging regulatory roles, and applications in protein engineering. Front Microbiol 14, 1305848. doi: 10.3389/fmicb.2023.1305848.
Wood, D. W., and Camarero, J. A. (2014). Intein applications: from protein purification and labeling to metabolic control methods. J Biol Chem 289, 14512-14519. doi: 10.1074/jbc.R114.552653.
Wood, D. W., Wu, W., Belfort, G., Derbyshire, V., and Belfort, M. (1999). A genetic system yields self-cleaving inteins for bioseparations. Nat Biotechnol 17, 889-892. doi: 10.1038/12879.
Woods, D., Vangaveti, S., Egbanum, I., Sweeney, A. M., Li, Z., Bacot-Davis, V., et al. (2020). Conditional DnaB Protein Splicing Is Reversibly Inhibited by Zinc in Mycobacteria. mBio 11, e01403-20. doi: 10.1128/mBio.01403-20.

1. A polypeptide having intein activity, comprising an amino acid sequence having at least about 80%, at least about 85%, at least about 90%, at least about 91%, at least about 92%, at least about 93%, at least about 94%, at least about 95%, at least about 96%, at least about 97%, at least about 98%, or at least about 99%, but less than 100%, sequence identity to the amino acid sequence of SEQ ID NO: 1 (TKΔE) or SEQ ID NO: 2 (TkPl), wherein the polypeptide has intein activity.

2. The polypeptide of claim 1, further comprising an A residue at a position corresponding to position 1 and/or a position corresponding to position 172 of SEQ ID NO: 1 or SEQ ID NO: 2.

3. The polypeptide of claim 1, further comprising an A residue at position 1 and/or at position 172 of SEQ ID NO: 1 or SEQ ID NO: 2.

4. The polypeptide of claim 1, wherein the polypeptide has at least 90%, but less than 100%, sequence identity to the amino acid sequence of SEQ ID NO: 1 or SEQ ID NO: 2.

5. The polypeptide of claim 1, wherein the polypeptide has at least 95%, but less than 100%, sequence identity to the amino acid sequence of SEQ ID NO: 1 or SEQ ID NO: 2.

6. The polypeptide of claim 1, wherein the polypeptide comprises, consists essentially of, or consists of:
(a) the amino acid sequence of SEQ ID NO: 1 with an A residue at positions 1 and/or 172; and
(b) the amino acid sequence of SEQ ID NO: 2 with an A residue at positions 1 and/or 172.

7. The polypeptide of claim 1, wherein:
(a) the polypeptide exhibits impaired splicing and N-terminal cleavage (NTC) at temperatures below 20° C.; and/or
(b) the polypeptide exhibits controllable splicing and N-terminal cleavage at temperatures ranging from about 20° C. to about 50° C.

8. A fusion protein comprising from N- to C-terminus an N-terminal extein polypeptide, an intein polypeptide, and a C-terminal extein polypeptide, wherein the intein polypeptide comprises the polypeptide having intein activity of claim 1.

9. The fusion protein of claim 8, wherein the residue at the −1 position of the N-terminal extein polypeptide is selected to control the rate of NTC, wherein:
(a) the −1 position of the N-terminal extein polypeptide is selected from a D, H, or K residue for fast NTC wherein at least 50% of the NTC occurs at 37° C. within about 2 hours;
(b) the −1 position of the N-terminal extein polypeptide is selected from a E, F, G, L, M, Q, R, W or Y residue for moderate NTC wherein at least 50% of the NTC occurs at 37° C. within about 6 hours; or
(c) the −1 position of the N-terminal extein polypeptide is selected from a A, C, I, N, P, S, T, or V residue for slow NTC wherein less than about 50% of the NTC occurs at 37° C. after about 6 hours.

10. The fusion protein of claim 8, wherein the residue at the −1 position of the N-terminal extein polypeptide is a D residue.

11. The fusion protein of claim 8, wherein the N-terminal extein polypeptide comprises a protein of interest.

12. The fusion protein of claim 8, wherein the N-terminal extein polypeptide comprises an N-terminal affinity tag.

13. The fusion protein of claim 8, wherein the C-terminal extein polypeptide comprises a reporter polypeptide.

14. An intein-based protein expression and purification system, comprising:
(a) the polypeptide having intein activity of claim 1;
(b) a host cell comprising an expression vector or construct encoding the polypeptide or the fusion protein for expression of a protein of interest; and
(c) a chromatography resin for purification of the protein of interest.

15. The system of claim 14, wherein the expression vector or construct is operably linked to an inducible promoter.

16. The system of claim 15, further comprising an inducer for promoting expression of the protein of interest.

17. The system of claim 13, wherein the chromatography resin comprises metal affinity chromatography beads and the N-terminal affinity tag comprises a His-tag.

18. The system of claim 14, further comprising a nucleophile to increase the rate of NTC.

19. A kit comprising the polypeptide having intein activity of claim 1, and instructions for using the polypeptide, the fusion protein, or the system for intein-based purification of a protein of interest.

20. A process for separating of a protein of interest on a chromatography resin, comprising:
(a) contacting the fusion protein of claim 8, with a chromatography resin to produce a chromatography resin-bound fusion protein, wherein contacting is performed at temperatures below 20° C. reduce or prevent NTC of the N-terminal extein polypeptide while the fusion protein remains bound to the chromatography resin;
(b) incubating the chromatography resin-bound fusion protein at temperature greater than or equal to 20° C. to initiate NTC of the N-terminal extein polypeptide and release the polypeptide having intein activity and C-terminal extein polypeptide so that the N-terminal extein polypeptide remains bound to the chromatography resin; and
(c) recovering the N-terminal extein polypeptide from the chromatography resin.


SEQUENCE LISTING (4 sequences)

SEQ ID NO: 1 - protein, 172 aa, Synthetic construct
CFAKDTKVYYENDTLVHFESIEDMYHKYASLGREVPFDNGYAVPLETVSVYTFDPKTGEVKRTKASYIYREKVEKLAEIRLSNGYLLRITLLHPVLVFRNGLQWVPAGMIKPGDLIVGIRSVPANATIELARRLEFHEVSSVEVVDYNDWVYDLVIPETHNFIAPNGLVLHN

SEQ ID NO: 2 - protein, 172 aa, Synthetic construct
CFAKDTKVYYENDTLVHFESIEDMYHKYASLGREVPFDNGYAVPLETVSVYTFDPKTGEVKRTKASYIYREKVEKLAEIRLSNGYLLRITLLHPVLVFRNGLQWVPAGMIKPGDLIVGIREEVLRRRIISKGELEFHEVSSVEVVDYNDWVYDLVIPETHNFIAPNGLVLHN

SEQ ID NO: 3 - protein, 482 aa, Synthetic construct
CFAKDTKVYYENDTLVHFESIEDMYHKYASLGREVPFDNGYAVPLETVSVYTFDPKTGEVKRTKASYIYREKVEKLAEIRLSNGYLLRITLLHPVLVFRNGLQWVPAGMIKPGDLIVGIRSVPANAATIEESEAYFLGLFVAEGTSNPLSITTGSEELKDFIVSFIEDHDGYTPTVEVRRGLYRILFRKKTAEWLGELATSNASTKVVPERVLNAGESAIAAFLAGYLDGDGYLTESIVELVTKSRELADGLVFLLKRLGITPRISQKTIEGSVYYRIYITGEDRKTFEKVLEKSRIKPGEMNEGGVGRYPPALGKFLGKLYSEFRLPKRDNETAYHILTRSRNVWFTEKTLSRIEEYFREALEKLSEARKALEMGDKPELPFPWTAITKYGFTDRQVANYRTRGLPKRPELKEKVVSALLKEIERLEGVAKLALETIELARRLEFHEVSSVEVVDYNDWVYDLVIPETHNFIAPNGLVLHN

SEQ ID NO: 4 - protein, 172 aa, Synthetic construct
CFARDTEVYYENDTVPHMESIEEMYSKYASMNGELPFDNGYAVPLDNVFVYTLDIASGEIKKTRASYIYREKVEKLIEIKLSSGYSLKVTPSHPVLLFRDGLQWVPAAEVKPGDVVVGVREEVLRRRIISKGELEFHEVSSVRIIDYNNWVYDLVIPETHNFIAPNGLVLHN